(19) United States
(12) Patent Application Publication
(10) Pub. No.: US 2026/0234206 A1
(43) Pub. Date: Aug. 13, 2026

(54) PROTEINS AND PROTEIN LIBRARIES

(71) Applicant: UNIVERSITY COLLEGE DUBLIN, NATIONAL UNIVERSITY OF IRELAND, DUBLIN, Belfield, Dublin (IE)

(72) Inventors: David O'CONNELL, Belfield, Dublin (IE); Sara LINSE, Belfield, Dublin (IE)

(21) Appl. No.: 18/708,542

(22) PCT Filed: Nov. 8, 2022
(86) PCT No.: PCT/EP2022/081181

§ 371 Date: May 8, 2024

(30) Foreign Application Priority Data
Nov. 8, 2021 (GB) 2116046.0

(51) Int. Cl. C07K 14/47; A61K 38/00; C12N 15/10; G01N 33/68

(52) U.S. Cl. CPC C07K 14/4728; C12N 15/1037; G01N 33/6845; A61K 38/00

(57) ABSTRACT

The present invention relates to proteins and protein libraries particularly for use in methods of screening to identify novel binding partners including diagnostic and therapeutic molecules.

### CROSS-REFERENCE TO RELATED APPLICATIONS

This application is the 35 U.S.C. § 371 national stage application of PCT Application No. PCT/EP2022/081181, filed Nov. 8, 2022, where the PCT claims priority to, and the benefit of, United Kingdom application entitled “Proteins and protein libraries” having serial no. GB2116046.0, filed Nov. 8, 2021, both of which are herein incorporated by reference in their entireties.

### REFERENCE TO SEQUENCE LISTING

The Sequence Listing submitted Jul. 29, 2025, as an Extensible Markup Language file named “P36828WO1 Sequence listing.xml,” created on Jul. 29, 2025, and having a size of 139,551 bytes is hereby incorporated by reference.

### TECHNICAL FIELD OF THE INVENTION

The present invention relates to proteins and protein libraries particularly for use in methods of screening to identify novel binding partners including diagnostic and therapeutic molecules.

### BACKGROUND TO THE INVENTION

The development of directed protein evolution technologies allied to more recent advances in high-throughput DNA synthesis has led to significant advances in the area of synthetic biology known as protein engineering. In clinical applications, fully engineered human or humanized monoclonal antibodies (mAbs) using protein evolution methods such as phage display have come to dominate the biotherapeutic pipeline in the past 30 years. Display technologies using libraries with very large sequence variability are a powerful method for the isolation of binder proteins to almost any antigen. Libraries of binder proteins based on human antibody variable gene sequences such as scFv (~25 kDa), Fabs to distantly related single domain VHH antibody-like binders from camelid immune libraries and nanobodies (~14 kDa) contact epitopes on antigens via the highly folded complementarity determining regions encoded by the variable genes. Entirely synthetic proteins that do not resemble immunoglobulin variable regions but that have been designed in silico have been developed as alternatives such as DARPins built on ankyrin repeat domains (~14 kDa), Avimers consisting of two or more peptide sequences of 30 to 35 amino acids each connected by linker peptides and Affimers derived from the cysteine protease inhibitor family of cystatins (12-14 kDa proteins that share the common tertiary structure of an alpha-helix lying on top of an anti-parallel beta-sheet). All of these engineered proteins scaffolds rely on a core structure with fixed positions providing a stable framework from which variable loops or surfaces can act as the binding interface with a specific target.

Existing protein libraries have numerous problems including the proteins being too large to bind to some protein interfaces, proteins being unstable, proteins being insoluble and proteins being cross-reactive with human proteins and other proteins of interest.

It is an object of the present invention to provide protein libraries particularly for use in methods of screening to identify novel diagnostic and therapeutic molecules which overcome the problems of existing protein libraries. The libraries of the invention provide a huge number of molecules which can be used in high-throughput screening against molecules associated with medical conditions and diseases. The present invention also provides novel proteins and peptides for use in the treatment and prevention of neurodegenerative diseases identified from such screening methods.

### SUMMARY OF THE INVENTION

In one aspect, the present invention relates to a double EF-hand protein comprising the amino acid sequence of any one of SEQ ID NOs: 2-11 or an amino acid sequence having at least about 50% sequence similarity or identity thereto, wherein X is any amino acid residue, wherein SEQ ID NOs: 7-11 include any one of inserts 1-20 and wherein the sequence similarity or identity is determined based on the amino acid residues of the constant region of SEQ ID NOs: 2-11. In one embodiment, the double EF-hand protein consists of said amino acid sequence. The SEQs and inserts are shown in Table 1.

In certain embodiments, the insert consists of 5 amino acids or 7 amino acids, or between 5 amino acids and 7 amino acids. The insert may optionally be flanked with up to 3 glycine residues. Preferably, the insert consists of 7 amino acids.

In a related aspect, the present invention relates to a double EF-hand protein comprising the amino acid sequence of any one of SEQ ID NOs: 2-11 or an amino acid sequence having at least about 50% sequence similarity or identity thereto, wherein X is any amino acid residue, wherein SEQ ID NOs: 7-11 include any one of inserts comprising any one of SEQ ID 12-39 and 74 and wherein the sequence similarity or identity is determined based on the amino acid residues of the constant region of SEQ ID NOs: 2-11. In one embodiment, the double EF-hand protein consists of said amino acid sequence. The SEQs and inserts are shown in Table 1.

In certain embodiments, the insert consists of 5 amino acids or 7 amino acids, or between 5 amino acids and 7 amino acids. The insert may optionally be flanked with up to 3 glycine residues. Preferably, the insert consists of 7 amino acids.

A double EF-hand protein is a protein comprising at least two EF-hands. An EF-hand is a helix-loop-helix structural domain or motif. The protein may further comprise one or more structural domains or motifs other than EF-hands. Human S100G is an example of a wild-type protein comprising two EF-hands. The full 79-residue amino acid sequence of wild-type human S100G is provided in SEQ ID NO: 1 (N-terminus→C-terminus). One EF-hand of wild-type human S100G (EF-hand 1) is formed by all or some of the amino acid residues occupying positions 1-47 of SEQ ID NO: 1 and the other EF-hand of wild-type human S100G (EF-hand 2) is formed by all or some of the amino acid residues occupying positions 48-79 of SEQ ID NO: 1.

SEQ ID NOs: 2-6 are base templates for side libraries based on the sequence of wild-type human S100G and SEQ ID NOs: 7-11 are base templates for loop libraries also based on the sequence of wild-type human S100G. The variable positions of SEQ ID NOs: 2-6 (represented by X) are located within EF-hand 1 (relative to wild-type human S100G) and the residues in these positions are exposed on a solvent-accessible surface of the protein. The 6 variable positions together represent the variable region of the side library base template sequences. SEQ ID NOs: 7-11 include an insert between EF-hand 1 and EF-hand 2 (relative to wild-type human S100G). The insertion comprises 3-7 variable positions (represented by X) which are optionally flanked by 1-3 glycine residues on each side. The flanks act as flexible hinges. The residues of the insertion protrude out from the protein. The 3-7 variable positions and the flanks (if present) together represent the variable region of the loop library base template sequences. The amino acid residues of SEQ ID NOs: 2-11 which are not within the variable region are within the constant region.

Amino acid sequences of the invention shown N-terminus → C-terminus. Boxed residues
represent variable positions in side libraries. <> represents the location of the insert in loop
libraries.
Wild-Type Human S100G (79 aa) (Uniprot P29377)
Underlined residues are examples of residues which can be subject to amino acid
substitutions in side and loop libraries. Residues in parentheses are examples of residues
which can be deleted in side and loop libraries.

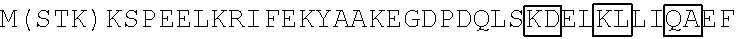

PSLLKGP<>NTLDDLFQELDKNGDGEVSFEEFQVLVKKISQ
(SEQ ID NO: 1)
Side Library Base Templates

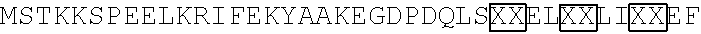

PSLLKGPNTLDDLFQELDKNGDGEVSFEEFQVLVKKISQ
(SEQ ID NO: 2)

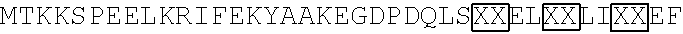

PSLLKGPNTLDDLFQELDKNGDGEVSFEEFQVLVKKISQ
(SEQ ID NO: 3)

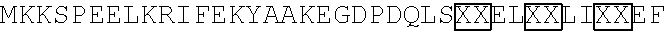

PSLLKGPNTLDDLFQELDKNGDGEVSFEEFQVLVKKISQ
(SEQ ID NO: 4)

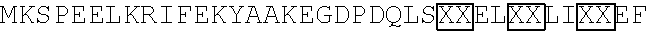

PSLLKGPNTLDDLFQELDKNGDGEVSFEEFQVLVKKISQ
(SEQ ID NO: 5)

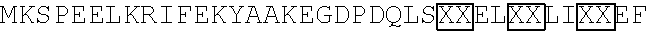

PSLLKGMSTLDDLFQELDKDGDGEVSFEEFQVLVKKISQ
(SEQ ID NO: 6)
Loop Library Base Templates
MSTKKSPEELKRIFEKYAAKEGDPDQLSKDELKLLIQAEF
PSLLKGP<>NTLDDLFQELDKNGDGEVSFEEFQVLVKKISQ
(SEQ ID NO: 7)
MTKKSPEELKRIFEKYAAKEGDPDQLSKDELKLLIQAEF
PSLLKGP<>NTLDDLFQELDKNGDGEVSFEEFQVLVKKISQ
(SEQ ID NO: 8)
MKKSPEELKRIFEKYAAKEGDPDQLSKDELKLLIQAEF
PSLLKGP<>NTLDDLFQELDKNGDGEVSFEEFQVLVKKISQ
(SEQ ID NO: 9)
MKSPEELKRIFEKYAAKEGDPDQLSKDELKLLIQAEF
PSLLKGP<>NTLDDLFQELDKNGDGEVSFEEFQVLVKKISQ
(SEQ ID NO: 10)
MKSPEELKRIFEKYAAKEGDPDQLSKDELKLLIQAEF
PSLLKGM<>STLDDLFQELDKDGDGEVSFEEFQVLVKKISQ
(SEQ ID NO: 11)
Loop Library Inserts
1) GGGXXXXXXXGGG
2) GGGXXXXXXGGG
3) GGGXXXXXGGG
4) GGGXXXXGGG
5) GGGXXXGGG
6) GGXXXXXXXGG
7) GGXXXXXXGG
8) GGXXXXXGG
9) GGXXXXGG
10) GGXXXGG
11) GXXXXXXXG
12) GXXXXXXG
13) GXXXXXG
14) GXXXXG
15) GXXXG
16) XXXXXXX
17) XXXXXX
18) XXXXX
19) XXXX
20) XXX

In one embodiment, the sequence similarity or identity to the amino acid sequence of any one of SEQ ID NOs: 2-11 is at least about 60%, about 70%, about 80%, about 90%, about 95%, about 97%, about 98% or about 99%. The sequence similarity or identity between two amino acid sequences can be determined by aligning the sequences and assessing the proportion of corresponding residues which have similar physicochemical properties (for sequence similarity) or which are identical (for sequence identity). Amino acid substitutions or deletions can be made in the constant region in order to improve or introduce desirable characteristics to the proteins while not affecting essential characteristics such as the formation of the EF-hands and the residues of the variable positions being exposed on a solvent-accessible surface of the protein (side libraries) or protruding from the protein (loop libraries).

Regardless of the extent of sequence similarity or identity, the protein encoded by any one of SEQ ID NOs: 2-11 will comprise two EF-hands. Table 2 outlines a number of possible positions in the constant regions at which amino acid substitutions may be made and also possible substitutions which may be made at these positions. An additional amendment which is possible is the deletion of one or more of the residues STK at positions 2-4 (relative to SEQ ID NO: 1).

TABLE 2
Example amino acid substitutions.

| Position | |
|---|---|
| (relative to SEQ ID NO: 1) | Substitution |
| 47 | P → M |
| 48 | N → S |
| 60 | N → D |

X can be any amino acid residue including a residue of an amino acid analogue, a synthetic amino acid, a non-natural amino acid or a natural amino acid. In one embodiment, X is any amino acid residue other than cysteine (C). Cysteine is preferably avoided in the variable region because of its ability to form disulphide bonds with other cysteine residues. In one embodiment, X is an amino acid residue selected from: alanine (A), arginine (R), asparagine (N), aspartic acid (D), glutamine (Q), glutamic acid (E), glycine (G), histidine (H), isoleucine (I), leucine (L), lysine (K), methionine (M), phenylalanine (F), proline (P), serine(S), threonine (T), tryptophan (W), tyrosine (Y) or valine (V).

In one embodiment, the double EF-hand protein is non-wild-type. In one embodiment, the double EF-hand protein is not wild-type human S100G. In one embodiment, the double EF-hand protein does not comprise the amino acid sequence of SEQ ID NO: 1.

In one embodiment, the double EF-hand protein is adapted to be displayed on the surface of a cell or on the surface of a virus. In one embodiment, the double EF-hand protein is adapted to be displayed on the surface of a bacteriophage. In one embodiment, the double EF-hand protein is fused with a viral coat protein. In one embodiment, the double EF-hand protein is fused with a bacteriophage coat protein.

In one aspect, the present invention relates to an S100G protein, wherein at least 1 amino acid residue in an EF-hand is different to the residue in the corresponding position of the human wild-type sequence. In one embodiment, 2, 3, 4, 5, 6 or more amino acid residues are different to the residue in the corresponding position of the human wild-type sequence. In one embodiment, the positions occupied by amino acid residues different to the residue in the corresponding position of the human wild-type sequence are in EF-hand 1. In one embodiment, the residues occupying the positions occupied by amino acid residues different to the residue in the corresponding position of the human wild-type sequence are on a solvent-accessible surface of the protein. In one embodiment, the positions at which the amino acid residues are different to the residue in the corresponding position of the human wild-type sequence correspond to one or more of positions 29, 30, 33, 34, 37 or 38 of the human wild-type sequence.

In one aspect, the present invention relates to an S100G protein, wherein the protein comprises a sequence insert between the two EF-hands. In one embodiment, the sequence insert consists of 3-13 or more amino acid residues. In one embodiment, at least 3 of the residues of the insert protrude from the surface of the protein. In one embodiment, the sequence insert is located between positions 47 and 48 relative to the human wild-type sequence.

In one aspect, the present invention relates to a nucleic acid encoding a protein disclosed herein. In some embodiments, the nucleic acid is DNA or RNA. In one embodiment, the nucleic acid is a vector. In one embodiment, the nucleic acid is adapted to display the protein on the surface of a cell or a virus such as a bacteriophage.

In one aspect, the present invention relates to a cell comprising a nucleic acid encoding a protein disclosed herein. In one embodiment, the cell is adapted to display the protein on the surface of the cell.

In one aspect, the present invention relates to a virus particle comprising a nucleic acid encoding a protein disclosed herein. In one embodiment, the virus particle is adapted to display the protein on the surface of the virus particle. In one embodiment, the virus is a bacteriophage.

A library is a collection of two or more library members wherein each library member is a substance or composition which is unique compared to the other members of the library. Library members may be proteins, nucleic acids, cells, virus particles, small molecules or compositions comprising one or more of proteins, nucleic acids, cells, virus particles and small molecules. The members of a library may be stored separately or together. A library may exist solely in silico.

Where a library comprises two or more protein members or two or more composition members comprising proteins, the following terminology may be used. A base template amino acid sequence is an amino acid sequence representing the constant positions and variable positions of at least a portion of the amino acid sequences of a number of proteins. Where the amino acid sequences of a number of proteins are aligned, the positions which are always occupied by the same residue can be termed constant positions and the positions which are not always occupied by the same residue can be termed variable positions. The constant positions and variable positions in a template sequence can collectively be termed the constant region and variable region, respectively. A number of proteins which share the same template amino acid sequence may be referred to as a sub-library. Unique proteins within such a sub-library may be referred to as variants. The maximum number of variants in a sub-library (V) can be calculated using the formula V=R^{P} wherein P represents the number of variable positions in the template sequence and R represents the number of different amino acid residues which can occupy each of the variable positions. For example, if a template amino acid sequence contains 3 variable positions which can each be occupied by 19 different amino acid residues, the maximum number of variants in a sub-library based on that template sequence is 193 (6, 859 variants).

In one aspect, the present invention relates to a library comprising two or more double EF-hand proteins of different amino acid sequences.

In one embodiment, the amino acid sequences of the double EF-hand proteins differ in 3, 4, 5, 6, 7 or more variable positions. In one embodiment, the variable positions are located within an EF-hand. In one embodiment, the variable positions located within an EF-hand are not located consecutively. In one embodiment, the variable positions are located between two EF-hands. In one embodiment, the variable positions located between two EF-hands are flanked by 1, 2, 3 or more glycine residues on each side. In one embodiment, the variable positions located between two EF-hands and the optional flanks are located consecutively. In one embodiment, the amino acid residues occupying the variable positions are exposed on a solvent-accessible surface of the protein in its tertiary structure or protrude from the surface of the protein in its tertiary structure. Such exposure or protrusion ensures that those residues can be involved in the binding interactions of the protein with other substances.

A sub-library based on a template sequence wherein the variable region is located in an EF-hand can be referred to as a side sub-library. A sub-library based on a template sequence wherein the variable region is located between two EF-hands can be referred to as a loop sub-library.

In one embodiment, the double EF-hand proteins comprise the amino acid sequence of any one of SEQ ID NOs: 2-11 or an amino acid sequence having at least about 50% sequence similarity or identity thereto, wherein X is any amino acid residue, wherein SEQ ID NOs: 7-11 include any one of inserts 1-20 and wherein the sequence similarity or identity is determined based on the amino acid residues of the constant region of SEQ ID NOs: 2-11. The embodiments disclosed above in relation to proteins of the invention such as the identity of X and the determination of sequence similarity or identity apply equally to proteins of libraries of the invention. In one embodiment, the double EF-hand proteins consist of said amino acid sequences.

In one embodiment, a library may comprise at least about 50%, about 60%, about 70%, about 80%, about 90%, about 95%, about 97% about 98%, about 99% or about 100% of the maximum number of variants of a sub-library. In one embodiment, a library may comprise proteins from two or more sub-libraries. In one embodiment, a library may comprise proteins from one or more side sub-libraries in addition to proteins from one or more loop sub-libraries.

In one embodiment, the proteins are displayed on the surface of cells or on the surface of virus particles. In one embodiment, the virus is a bacteriophage.

In one aspect, the present invention relates to nucleic acids encoding the proteins of a library disclosed herein, cells comprising nucleic acids encoding the proteins of a library disclosed herein or virus particles comprising nucleic acids encoding the proteins of a library disclosed herein. In one embodiment, the virus is a bacteriophage.

In one aspect, the present invention relates to a method of producing or expanding a library, the method comprising the step of collecting two or more double EF-hand proteins of different amino acid sequences or nucleic acids encoding such proteins. In one embodiment, the method further comprises a preceding step of synthesising such proteins or nucleic acids. In one embodiment, the proteins or nucleic acids of the library are proteins or nucleic acids of any library disclosed herein.

In one aspect, the present invention relates to a method of screening, the method comprising: (a) exposing a target molecule to the proteins of a library disclosed herein; and (b) assessing the binding affinity between the target molecule and one or more of the proteins. In one embodiment, the method additionally comprises the step (c) of selecting one or more proteins which bind to the target molecule with an affinity at or above a selected level.

In some embodiments, the proteins of the library are displayed on the surface of cells, on the surface of virus particles or on the surface of yeast particles (yeast-display). In one embodiment, the virus is a bacteriophage (phage-display). In other embodiments, the method of screening uses ribosome-display, split-GFP screening or mRNA-display.

Binding affinity between a target molecule and proteins of a library may be measured by any suitable means such as surface plasmon resonance, isothermal calorimetry, microscale thermophoresis, microfluidics diffusional sizing and alternative in-solution kinetic analyses.

In one embodiment, the target molecule is associated with a medical condition or disease such as cancer or a neurodegenerative disease such as Alzheimer's disease. For instance, the target molecule may be present at a greater level or in a particular form in individuals who have been diagnosed with a particular medical condition or disease compared to individuals who have not been diagnosed with the medical condition or disease.

In one aspect, the present invention relates to a pharmaceutical composition comprising a protein disclosed herein or a nucleic acid encoding such a protein. In one embodiment, the pharmaceutical composition is for use in the prevention or treatment of a medical condition or disease.

In one aspect, the present invention relates to a method for the prevention or treatment of a medical condition or disease, the method comprising the administration of the pharmaceutical composition disclosed herein to a subject.

In one aspect, the present invention relates to the use of a protein or nucleic acid disclosed herein in the manufacture of a medicament for the prevention or treatment of a medical condition or disease.

The pharmaceutical composition or medicament may further comprise one or more selected from: one or more pharmaceutically acceptable carriers, one or more pharmaceutically accepted excipients and one or more further active ingredients. The pharmaceutical composition or medicament may be adapted to diffuse across the blood-brain-barrier. The pharmaceutical composition or medicament may be administered to subjects by any suitable means and in any dosage regimen which is sufficient to achieve the desired therapeutic or prophylactic effects, and which is safe to the subject.

In one embodiment, the medical condition or disease is cancer or a neurodegenerative disease. In one embodiment, the neurodegenerative disease is a neurodegenerative disease associated with the aggregation of AB which means any neurodegenerative disease the presence of which in subjects is correlated with excess aggregation of AB. In one embodiment, the neurodegenerative disease is Alzheimer's disease.

In one embodiment, the protein in the pharmaceutical composition or medicament comprises or consists of the amino acid sequence of any one of SEQ ID NOs: 2-6 wherein the variable positions are occupied by VI-WI-DD or the amino acid sequence of any one of SEQ ID NOs: 7-11 with any one of inserts 1, 6, 11 or 16 wherein the variable positions are occupied by YLTIRLM (SEQ ID NO: 74), or an amino acid sequence having at least 50% sequence similarity or identity thereto, and wherein the sequence similarity or identity is determined based on the amino acid residues of the constant region of SEQ ID NOs: 2-11.

In another embodiment, the protein in the pharmaceutical composition or medicament comprises or consists of the amino acid sequence of any one of SEQ ID NOs: 2-6 wherein the variable positions are occupied by VI-WI-DD or the amino acid sequence of any one of SEQ ID NOs: 7-11 with any one of inserts 1, 6, 11 or 16 wherein the variable positions are occupied by any one of SEQ NOs: 12-39, or an amino acid sequence having at least 50% sequence similarity or identity thereto, and wherein the sequence similarity or identity is determined based on the amino acid residues of the constant region of SEQ ID NOs: 2-11.

In one aspect, the present invention relates to a protein or peptide comprising or consisting of the amino acid sequence YLTIRLM (SEQ ID NO: 74). The amino acid sequence may be flanked with up to 3 glycine residues on either side of the sequence. In one embodiment, the protein or peptide is for use in the prevention or treatment of a neurodegenerative disease associated with the aggregation of amyloid β peptide (AB) such as Alzheimer's disease.

In another aspect, the present invention relates to a protein or peptide comprising or consisting of one or more amino acid sequences selected from: SFTFRAA (SEQ ID NO: 12), SLNTTFP (SEQ ID NO: 13), SSSLPFP (SEQ ID NO: 14), NASFRTP (SEQ ID NO: 15), NFAIPLL (SEQ ID NO: 16), SVTSPFT (SEQ ID NO: 17), SFNFTIA (SEQ ID NO: 18), SLNVTFV (SEQ ID NO: 19), AIDSPFV (SEQ ID NO: 20), YVYLQVV (SEQ ID NO: 21), YFSFRPL (SEQ ID NO: 22), SVYVRLV (SEQ ID NO: 23), SASFRLL (SEQ ID NO: 24), SVSIKVK (SEQ ID NO: 25), YLYVTLV (SEQ ID NO: 26), YLYLRIM (SEQ ID NO: 27), SFYVRFL (SEQ ID NO: 28), TLSIRVA (SEQ ID NO: 29), DVDLKPL (SEQ ID NO: 30), APSARLE (SEQ ID NO: 31), TVDTKLL (SEQ ID NO: 32), INLAF (SEQ ID NO: 33), VKGSA (SEQ ID NO: 34), RRFVN (SEQ ID NO: 35), SSTTS (SEQ ID NO: 36), SLTLT (SEQ ID NO: 37), RLYLT (SEQ ID NO: 38) and VGSMF (SEQ IN NO; 29). The one or more amino acid sequences may be flanked with up to 3 glycine residues on either side of the sequences. In one embodiment, the protein or peptide is for use in the prevention or treatment of a neurodegenerative disease associated with the aggregation of amyloid β peptide (AB) such as Alzheimer's disease.

### BRIEF DESCRIPTION OF THE DRAWINGS

Embodiments of the present invention will now be described, by way of example only, with reference to the accompanying figures, in which:

FIG. 1 shows structural models and the amino acid sequences of a side library base template (top) including a 6-residue randomised patch at the side of EF-hand 1 (around 5.107 variants) and a loop library base template (bottom) including an insert with a 7-residue randomised segment with glycine flanks inserted between EF-hand 1 and EF-hand 2 (around 9.108 variants). The crystal structure of the base templates (3icb.pdb), is shown as space filling models (left) and ribbon (right) with EF-hand 1 shown in grey and EF-hand 2 in dark grey. The loop insertion between residues 43 and 44 is shown in white. The variable residues that are randomized over 19 residue types, excluding cysteine, are shown in black in the structural models and are indicated by X in the sequences.

FIG. 2 is a Venn diagram of sequence analysis of round 2 of selection across three antigens with two libraries. Sequences that were present more than 100 times in each round were included for analysis. For the loop library, the calmodulin binders (CaM) from round 2 were compared with round 3 sequences for IgG and HSA. For the side library, round 3 sequences were compared for all three antigens.

FIG. 3 is a series of chromatograms of ion exchange chromatography purification of SXkmer protein. (LEFT) DEAE sepharose chromatography anion exchange resin is used to bind positively charged proteins in a calcium free EDTA buffer with elution fractions (F11, 12, 13 & 16) proceeding to a second ion exchange. (RIGHT) Q HP strong anion exchange resin is used with a calcium containing buffer for purification of eluted fractions (F21-26).

FIG. 4 is a series of graphs representing biophysical analysis of SXkmer binding. (LEFT) Surface plasmon resonance sensorgram of 100 nM anti-Calmodulin SXkmer A (KMRPGVN) binding to immobilised calmodulin. (RIGHT) Thermophoresis analysis of the binding of Alexaflour488 labelled SXkmer A to free calmodulin in solution.

FIG. 5 is a series of graphs showing the frequency of occurrence of the top 500 hits from the 3rd round of selection of A) the loop library and B) the side library versus Aβ42 monomer, Aβ40 monomer, Aβ42 fibril and Aβ40 fibril.

FIG. 6 is the amino acid sequences of the two SXkmers produced in this work as binders of A) Aβ42 monomer or oligomer, SXkmer-YLTIRLM, and B) Aβ40 monomer or oligomer, SXkmer-VI-WI-DD. Bold letters are used to indicate the amino acid residues found at the positions which were randomized in the respective library (See FIG. 1) and the three glycine residues introduced at each side of the loop insert. (C-D) Alignment clusters showing all sequences with a score of at least 30 (C, loop) or 27 (D, side). The score for 100% identify would be 63 (loop) or 56 (side).

FIG. 7 is a series of graphs and electrophoretograms showing the purification of the YLTIRLM loop library sequence. (A) Elution of 5 mL QHP anion exchange column in 10 mM Tris/HCL, 1 mM EDTA with a linear salt gradient from 0 to 500 mM NaCl. The absorbance at 280 nm (blue) and 255 nm (green) and the conductivity (red) are shown. The vertical dashed lines indicate the fractions collected for the next step. (B) Elution of 5 mL QHP anion exchange column in 10 mM Tris/HCL, 1 mM CaCl2 with salt gradient from 0 to 300 mM NaCl. The absorbance at 280 nm (blue) and 255 nm (green) and the conductivity (red) are shown. The vertical dashed lines indicate the fractions collected for the next step. (C) Elution of Superdex 75 (10/300 mm) size exclusion column in 20 mM sodium phosphate, 0.2 mM EDTA, pH 8.0. The absorbance at 280 nm (blue) and the conductivity (red) are shown. The vertical dashed lines indicate the fractions collected for kinetics and binding analyses. (D) SDS PAGE (4-20% gradient gel in 0.1 M Tris/tricine buffer) analysis of eluted fractions. L=low Mw standard (green band=10 kDa), S=high Mw standard (green band=10 kDa). Lane 1=boiled cells supernatant, 2=eluted fractions from first QHP column in EDTA, 3-5=eluted fractions from second QHP column in CaCl2 (nr 4 was applied to SEC column), 6-9=eluted fractions from SEC.

FIG. 8 is a series of graphs and a photograph showing the effect of SXkmer-YLTIRLM on Aβ42 aggregation kinetics. (A-C) Aggregation kinetics of 3 μM Aβ42 in the absence and presence of SXkmer-YLTIRLM (separate shades of grey for each concentration as indicated in μM) in 10 mM Tris, 150 mM NaCl, 100 μM CaCl2, pH 8.0 monitored by ThT fluorescence. The data were fitted three times assuming selective reduction of the rate constant for primary nucleation (A), secondary nucleation (B) and elongation (C). (D-E) seeded aggregation. Data obtained without (light grey) and with (grey) SXkmer-YLTIRLM for reactions starting from 2 μM Aβ42 monomer supplemented with 1% seed (D) or 30% seed (E). (F) Cryo-EM image of Aβ42 fibrils formed in the presence of SXkmer-YLTIRLM.

FIG. 9 is a series of graphs showing aggregation kinetics of 15 μM Aβ40 in the absence and presence of SXkmer-VI-WI-DD (separate shades of grey for each concentration as indicated in the left panel in μM) in 10 mM Tris, 150 mM NaCl, 100 μM CaCl2, pH 7.4 monitored by ThT fluorescence. The data were fitted three times assuming selective reduction of the rate constant for primary nucleation (left), secondary nucleation (middle) and elongation (right).

FIG. 10 is a graph showing SPR analysis. The top two traces show examples of sensorgrams from two flow cells immobilized with Aβ42 fibrils with an initial injection of 150 μl of 10 μM Aβ42 monomer, followed by an injection of 150 μl of 1.0 μM SXkmer-YLTIRLM, and finally buffer flow. The middle two traces show examples of sensorgrams from the injection of 250 μl of 1.0 μM SXkmer-YLTIRLM over immobilized Aβ42 fibrils that had been equilibrated for 2 h past immobilization, followed by buffer flow. The bottom trace shows an example of a sensorgram from the injection of 250 μl of 1.0 μM SXkmer-YLTIRLM over immobilized monomer followed by buffer flow. Blank traces from flow cells with no coupled Aβ42 are subtracted from all traces before plotting.

FIG. 11 is a graph showing the effect of the synthetic peptide YLTIRLM on Aβ42 aggregation kinetics. Aggregation kinetics of 3 μM Aβ42 in the absence and presence of YLTIRLM (separate shades of grey for each concentration as indicated in μM) in 20 mM sodium phosphate, 0.2 mM EDTA, pH 8.0 monitored by ThT fluorescence. The data were fitted assuming selective reduction of the rate constant for secondary nucleation.

FIG. 12 is a collection of graphs showing in-solution studies of SXkmer-YLTIRLM interaction with species withdrawn from an ongoing Aβ42 aggregation reaction. (A) Examples of fluorescence emission spectra for SXkmer-YLTIRLM-Alexa488 mixed with samples withdrawn from reactions of 9:1 Aβ42:Aβ42-Alexa555. The “sum” spectrum (black) is obtained by summing the spectrum of SXkmer-YLTIRLM-Alexa488 alone with that of Aβ42:Aβ42-Alexa555 alone. (B) FRET efficiency calculated from the fluorescence emission at 522 nm (i.e., from the donor SXkmer-YLTIRLM-Alexa488) shown as average and SD over three replicates. (C) Apparent hydrodynamic radius of Alexa-647-labeled species as a function of time of withdrawal from the ongoing Aβ42 reaction. (D) Examples of turbidity and ThT fluorescence traces from ongoing Aβ42 aggregation reactions. The samples followed by turbidity were used to acquire the data shown in panels A-C.

FIG. 13 is two graphs that show the results of continuous in-solution studies of SXkmer-YLTIRLM interaction with Aβ42 during an on-going Aβ42 aggregation reaction. A) Examples of fluorescence emission spectra for 0.1 μM SXkmer-YLTIRLM-Alexa488 mixed with 9 μM Aβ42 and 1 μM Aβ42-Alexa555, initially (0 h) and at three time points (10, 20 and 60 h) during an ongoing reaction. B) Relative fluorescence emission at 566 and 522 nm (red) and static light scattering (black) as a function of reaction time.

FIG. 14 is an image of two gels produced by a two-step ion exchange process. The first step involves ion exchange in EDTA (top panel) and the second step involves ion exchange in a Ca⁺ buffer (bottom panel).

FIG. 15 is a representation of the SXkmer YLTIRLM library development.

FIG. 16 is two images of gels produced by the expression of the various SXkmers found in the new YLTIRLM SXkmer library.

FIG. 17 is three graphs that show the AB Aggregation after exposure to the “YVYLQWV” (SEQ ID NO: 21) 7-mer SXkmer which has either been purified by 30k elution, size exclusion chromatography (SEC) or SEC followed by a freeze-thaw cycle.

FIG. 18 is three graphs that show the AB Aggregation after exposure to the “INLAF” (SEQ ID NO: 33) 5-mer SXkmer which has either been purified by 30k elution, size exclusion chromatography (SEC) or SEC followed by a freeze-thaw cycle.

FIG. 19 is a collection of graphs showing the Aβ Aggregation Inhibition over time with exposure to the 7-mer SXkmers included in the new focused library at a range of concentrations.

FIG. 20 is a collection of graphs showing the Aβ Aggregation Inhibition over time with exposure to the 5-mer SXkmers included in the new focused library at a range of concentrations.

### DETAILED DESCRIPTION OF THE INVENTION

### Example 1—Design & Application of Libraries

### Introduction

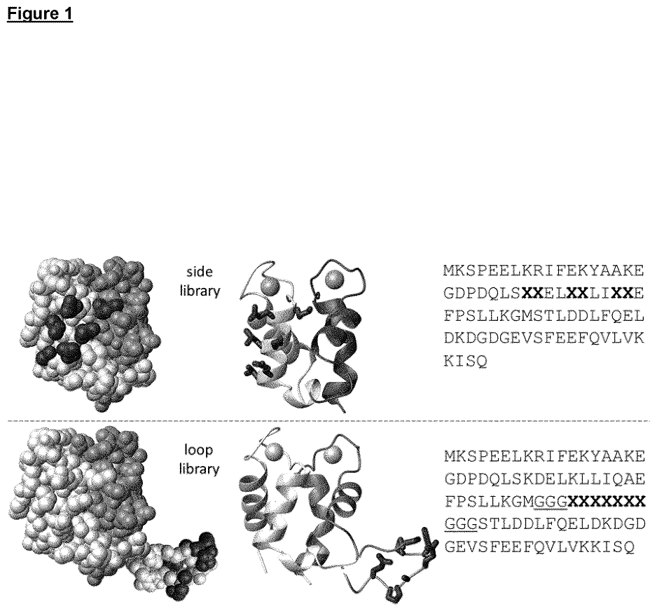

In the present study we have produced two phage-display libraries for selection of binding proteins built on a novel scaffold based on human S100G (also known as calbindin D9k). S100G is a tightly folded single-domain protein composed of two EF-hands (EF1 and EF2). S100G is an ideal scaffold because of its resistance towards proteolysis and denaturation. With bound Ca2+, the protein can be boiled in 8 M urea. Another desirable feature is its remarkable inertness to protein-protein interaction. S100G is a highly soluble protein with a highly hydrophilic surface, which has been employed as a negative control in protein array screening studies. The protein can be reconstituted with high-affinity from its EF-hands, providing a basis for a Ca2+-dependent purification tool and is highly tolerant to loop insertions between the EF-hands. S100G is therefore an attractive new scaffold for building target recognition functionality. Two libraries were constructed in the form of a side library with a 6-residue variable patch on the surface of EF1, and a loop library with a 7-residue variable loop insertion between EF1 and EF2 (FIG. 1), which we have termed SXkmer libraries.

### Methods

### Phage Display Experiment for the Selection of Binding Candidates

Six selections of binders were set up in parallel over three rounds in buffer A. Side and loop libraries (1 mL containing ca. 10¹² phage particles) were separately incubated with the IgG-coated immunotubes at 100 μg/mL for 1 hour at room temperature. Supernatants containing unbound phage were then added to a second pair of immunotubes coated with HSA at 50 μg/mL and post-incubation the supernatants from these selections were added to a pair of immunotubes coated with 10 μg/mL of CaM. After incubation with the libraries or amplified libraries for 1 h, the immunotubes were washed 10-20 times with buffer A. Phage were eluted through incubation with 500 μl 0.1 M Triethylamine, pH 2.2 for 5 min, and the eluates neutralized by adding to 1 ml 1 M Tris pH 7.4. Amplification in Tg1 E. coli was used to produce six enriched libraries after each round.

### Next Generation Sequencing (NGS)

The Tg1 cells infected with the third round eluates were used to prepare plasmids from cells pelleted from 2 mL ON culture using a plasmid purification kit (Illustra). The purified plasmids were used as templates in PCR reactions with the following primers: S100Gfor (GGCCATGGCCAAATCTCC (SEQ ID NO: 51)) and S100Grev (CCAGTTCCTGGAACAGGTC (SEQ ID NO: 52)) yielding PCR products of 170 bp for side library members and 209 bp for loop library members. NGS of the PCR products, to yield 5×10⁶ reads using the same two primers as above, was purchased from Eurofins.

### Bioinformatics and Sequence Alignment

The results of NGS (merged read files in fatsq.bz2 format) were analysed to provide text files containing all sequences that occurred at least 100 times. Each of the first 500 sequences (or as many as found if fewer than 500) was aligned against each of the other 499 sequences from the same selection and an alignment score calculated using a scoring matrix with scores between 9 (identity) and 0 (no resemblance) for each possible residue-residue comparison, to produce lists of alignment above a chosen cut-off. Genevenn (http://genevenn.sourceforge.net/) was used to compare sequences from the different rounds of selection with both libraries and three antigens.

### Results

### Immobilization and Selection of Binders

The two SXkmer phage display libraries, loop and side (FIG. 1) were used in three rounds of selection against human IgG, HSA and CaM immobilised on immunotubes. The unbound phage in the supernatant of the IgG selection in the first round was incubated with HSA and the unbound phage from this selection incubated with CaM in round 1.

### Next Generation Sequencing (NGS)

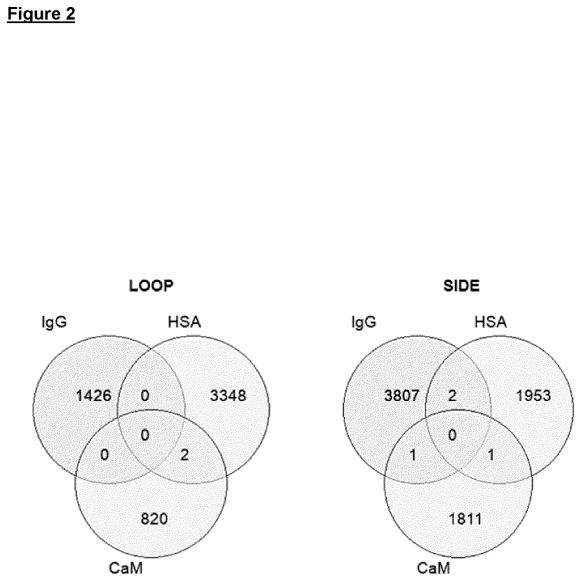

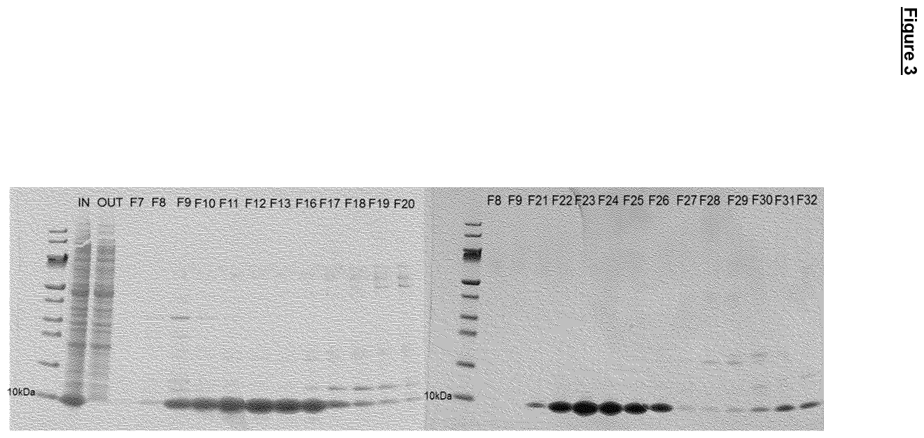

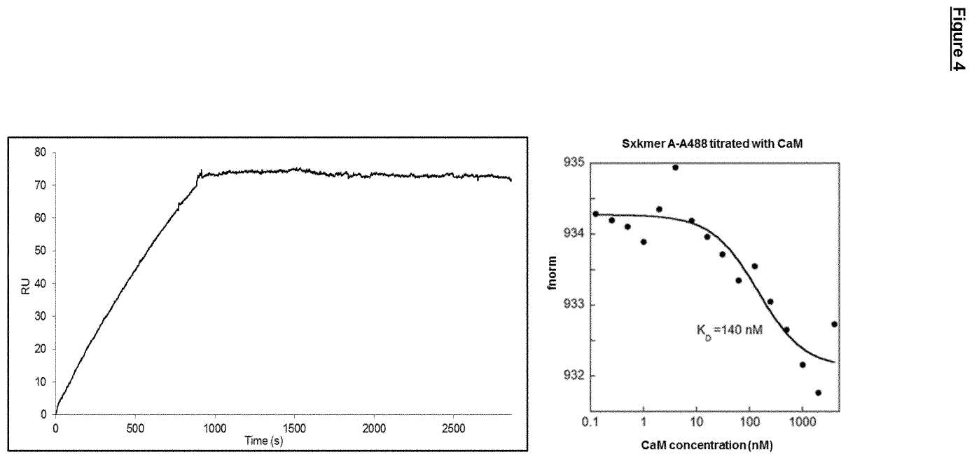

The eluates after rounds two and three of selection against each antigen were subjected to PCR using primers inside the constant region of S100G followed by NGS to provide information on the retrieved side and loop sequences. Round 3 of the CaM selection failed to give a single PCR product, with a ladder of bands, and was subsequently excluded from NGS sequencing. The distribution of sequences and the number of overlapping sequences across antigens after two rounds of selection are shown in FIG. 2. Sequences were selected from round 3, which had also been identified in round 2 for each antigen and two representative sequences from each selection were used. Two sequences from round 2 of the CaM selection were chosen separately (Table 3). Sequences that had a high similarity score from multiple sequence alignments were chosen for synthesis and subsequent cross reactivity analysis. The frequency of occurrence of the top 500 hits in each case is displayed in FIG. 3.

TABLE 3
Sequences selected for synthesis and functional analysis. Two
sequences from selections identified in the top 20 most frequent
sequences in round 3 that were also identified in round 2 were
chosen for synthesis.

| | Overlap | #Rd2 seqs in | | SEQ ID | | SEQ ID |
|---|---|---|---|---|---|---|
| Selection | R2/R3 | Rd3 Top 20 | Seq 1 | NO: | Seq 2 | NO: |
| Loop IgG | 21930 | 14/20 | QPVVRTN | 45 | HFQWHPT | 48 |
| Loop Alb | 394 | 2/20 | YTNAISD | 46 | WHHTRLE | 49 |
| Loop CaM | nd | nd | KMRPGVN | 47 | AVHHMPF | 50 |
| Side Alb | 217 | 11/20 | QE-EA-DH | 93 | FF-NQ-FS | 95 |
| Side CaM | 941 | 20/20 | SA-NS-VA | 94 | IQ-KY-YH | 96 |

### Production of Candidate SXkmers

The loop and the side patch sequences were reintroduced into human S100G and expressed as free proteins, SXkmers. The proteins were found to be expressed at a very high yield of ca. 1 g/L (SXkmer-Loop) and 1 g/L (SXkmer-Side) and were purified using sonication, boiling, two anion exchange steps in EDTA and Ca2+, respectively, and size exclusion chromatography (FIG. 3).

### Binding Kinetics

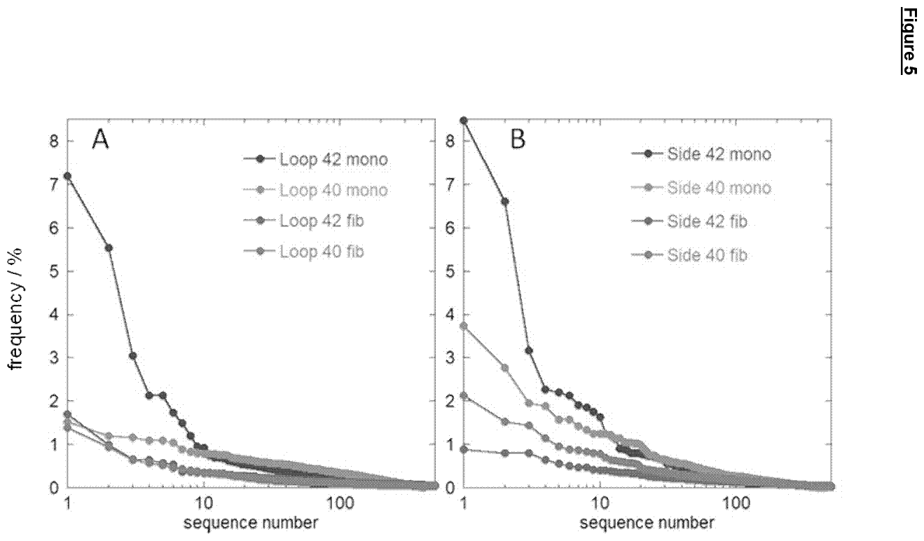

The binding of the Loop calmodulin binder SXkmer A (KMRPGVN (SEQ ID NO: 47) to immobilised IgG, HSA and CaM was studied in a flowpath of Fc4-1/Fc3-1/Fc2-1 (FIG. 4 LEFT). No binding was seen to either IgG or HSA in flow cells Fc4 and Fc3. The binding data were analysed using BiaEvaluate software and shows slow dissociation kinetics with a kd (1/s) of approximately 1×10⁻⁶. Microscale thermophoresis was used to estimate the in-solution binding kinetics to free calmodulin and the fitted KD value was calculated to be approximately 140 nM (FIG. 4 RIGHT).

### Human Microarray Specificity Analysis

High content human protein microarray specificity analysis was performed. Purified anti-CaM and anti-IgG SXKmer proteins were labelled with AlexaFlour 647 and incubated on HuProt human protein microarrays. Labelled SXkmer with GGG-XXXXXXX-GGG loop was used as a negative control.

Coincubation & SEC for in-Solution Study

Anti-CaM+CaM followed by SEC and PAGE (non-denaturing) and agarose gel in calcium were used.

### Conclusions

It was found that engineered protein scaffolds based on human S100G have the potential to expand the therapeutic and diagnostic landscape for complex protein targets because they have desirable physical characteristics enabling binding to complex protein targets not easily targeted by other protein formats.

The proteins of libraries based on S100G are small enough to bind to many protein interfaces which are currently difficult to access, are highly stable, are soluble and are not cross-reactive with human proteins and other proteins of interest. These libraries represent a huge reservoir of molecules which can be screened to find potential new therapeutic and diagnostic molecules.

### Example 2—Screening & Verification of Aβ Aggregation Inhibitors

### Introduction

Alzheimer's disease (AD) is a devastating neurodegenerative disease for which there is currently no cure (Hebert 2013; Selkoe 2013). The pathology of AD has been linked to the aggregation of two proteins: amyloid β peptide (Aβ) and the protein tau (Hardy 1992). Both proteins are found to exist as monomers, oligomers of various sizes (some of which are referred to as protofibrils), as well as highly ordered amyloid fibrils (Walsh 2002; Colvin 2016; Wälti 2016; Fitzpatrick 2017). According to the current consensus in the field, monomers and fibrils have relatively low toxicity to neurons compared to oligomeric reaction intermediates. However, the aggregation process of Aβ is autocatalytic and a majority of oligomers are generated from monomers on the surface of fibrils (Cohen 2013). The generation of new aggregates is dominated by secondary nucleation on the side of fibrils, which has a much lower energy barrier than primary nucleation in solution (Cohen 2018). Oligomers are transient species that may either convert to fibrils or dissociate into monomers that may undergo new cycles of oligomer formation and conversion (Michaels 2020).

High affinity binders for Aβ monomers and aggregates have distinct effects on the aggregation process and the evolution of toxicity (Arosio 2016). Inhibitors of secondary nucleation may limit the concentration of oligomers during the reaction and the toxicity caused to neurons in brain tissue (Cohen 2015; Munke 2017; Linse 2020). Inhibitors of primary nucleation may instead delay the onset of aggregation, and although the generation of toxicity is not reduced it may be greatly delayed (Månsson). Binding proteins for aggregates as well as monomers may thus provide valuable tools for novel therapeutics and diagnostics.

Side and loop libraries were used to select binders for Aβ40 and Aβ42 in the form of monomers and fibrils. In the latter case, fibril binders were enriched after depletion of monomer binders in an attempt to derive conformation-specific binders in analogy with previous work (Munke 2017). The eluates after three rounds of selection were subjected to next generation sequencing (NGS) for the identification of prominent sequences and sequence clusters, followed by expression and purification of candidates as free S100G proteins. Their activity as aggregation inhibitors was assayed using a thioflavin-T fluorescence using non-seeded and seeded samples, and the interaction with Aβ was analysed using microfluidics diffusion sizing, thermophoresis and surface plasmon resonance.

### Methods

### Expression and Purification of Aβ40 and Aβ42

Aβ (M1-40), here called Aβ40, and Aβ (M1-42), here called Aβ42, were recombinantly expressed in E. coli from PetSac plasmids containing synthetic genes with E. coli optimized codons, and purified from inclusion bodies using sonication, ion exchange and two rounds of size exclusion chromatography, and the isolated monomers stored as lyophylized aliquots as described (Walsh 2002; Cohen 2013; Linse 2020).

### Preparation of Monomers and Fibrils

Purified aliquots of Aβ40 and Aβ42 were separately dissolved in 1 mL 6M GuHCl and monomer isolated using size exclusion chromatography on a 10/300 Superdex 75 column (GE Healthcare) in 20 mM sodium phosphate, 0.2 mM EDTA, pH 8.0 (Aβ42) or pH 7.4 (Aβ40) in low-binding tubes (Axygen) on ice. The peptide concentration was determined by the integrated absorbance of the collected fraction using 8280=1400 l mol⁻¹ cm⁻¹. Fibrils were prepared from the monomer solutions at quiescent condition at 37° C. in multiple wells of a PEG-ylated polystyrene 96-well plate (Corning 3881) with thioflavin T (ThT) in some wells. The ThT fluorescence was monitored through the bottom of the plate using a BMG Optima plate reader with excitation at 440 nM and emission at 480 nM until reaching the ThT plateau. Fibrils were collected from wells without ThT.

### Coupling to Silica Nanoparticles

Silica nanoparticles were washed in 100 mM MES, pH 5.5, and activated by a mixture containing 0.2 M EDC and 50 mM NHS in water. Monomers or fibrils were diluted in 10 mM sodium acetate pH 3.0, and incubated with the activated particles for 30 minutes, followed by blocking with 1 M ethanolamine and washing three times with 10 mM Tris/HCl, 150 mM NaCl, 100 μM CaCl₂), pH 7.5, 0.1% Tween20 (buffer A). After each step, the nanoparticles were pelleted by centrifugation.

### Phage Display Experiment for the Selection of Binding Candidates

Eight selections of binders were set up in parallel over three rounds in buffer A. Side and loop library (0.5 mL containing ca. 10¹² phage particles), was separately incubated with the nanoparticle-conjugated Aβ40 or Aβ42 in the form of monomers or fibrils (Table 3). BSA was added at 2 mg/mL as a blocker of the particles before the third round of selection. After incubation with the libraries or amplified libraries for 1 h, the nanoparticles were washed ten times with buffer A. After each incubation and washing step, the nanoparticles were pelleted by centrifugation. Phage were eluted through incubation with 100 μl 0.1 M HCl/glycine, pH 2.2 for 60 min, nanoparticles pelleted and the supernatant neutralized by adding 20 μl 1 M Tris pH 9.1. Amplification in Tg1 E. coli was used to produce eight enriched libraries after each round according to the Tomlinson protocol.

### Next Generation Sequencing (NGS)

The Tg1 cells infected with the third round eluates were used to prepare plasmids from cells pelleted from 2 mL ON culture using a plasmid purification kit (Illustra). The purified plasmids were used as templates in PCR reactions with the following primers: S100Gfor (GGCCATGGCCAAATCTCC (SEQ ID NO: 51)) and S100Grev (CCAGTTCCTGGAACAGGTC (SEQ ID NO: 52)) yielding PCR products of 170 bp for side library members and 209 bp for loop library members. NGS of the PCR products, to yield 5×10⁶ reads using the same two primers as above, was purchased from Eurofins.

### Bioinformatics and Sequence Alignment

The results of NGS (merged read files in fatsq.bz2 format) were analyzed to provide text files containing all sequences that occurred at least 100 times. Each of the first 500 sequences (or as many as found if fewer than 500) was aligned against each of the other 499 sequences from the same selection and an alignment score calculated using a scoring matrix with scores between 9 (identity) and 0 (no resemblance) for each possible residue-residue comparison, to produce lists of alignment above a chosen cut-off.

### Results

### Immobilization and Selection of Binders

The two Sxmer phage display libraries, loop and side (FIG. 1) were used in three rounds of selection with an aim to retrieve both monomer and fibril-binders to Aβ40 as well as Aβ42. For this aim, recombinantly expressed and purified monomers of Aβ40 and Aβ42 were separately coupled to silica nanoparticles. Monomer samples were also used to generate Aβ40 and Aβ42 fibrils, as validated using ThT fluorescence, which were separately coupled to silica nanoparticles. The selection of monomer binders used the monomer beads only. The selection of fibril binders was preceded by monomer depletion on monomer-coupled beads before incubation with fibril-coupled beads.

### Next Generation Sequencing (NGS)

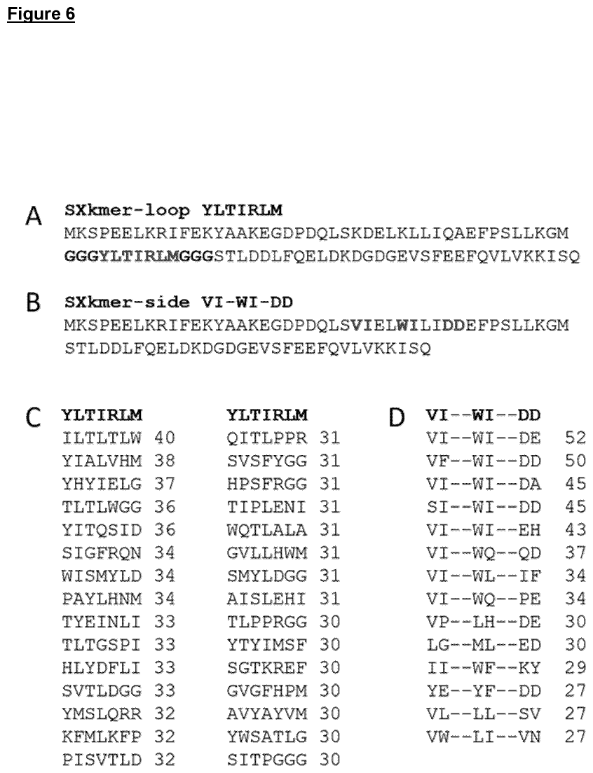

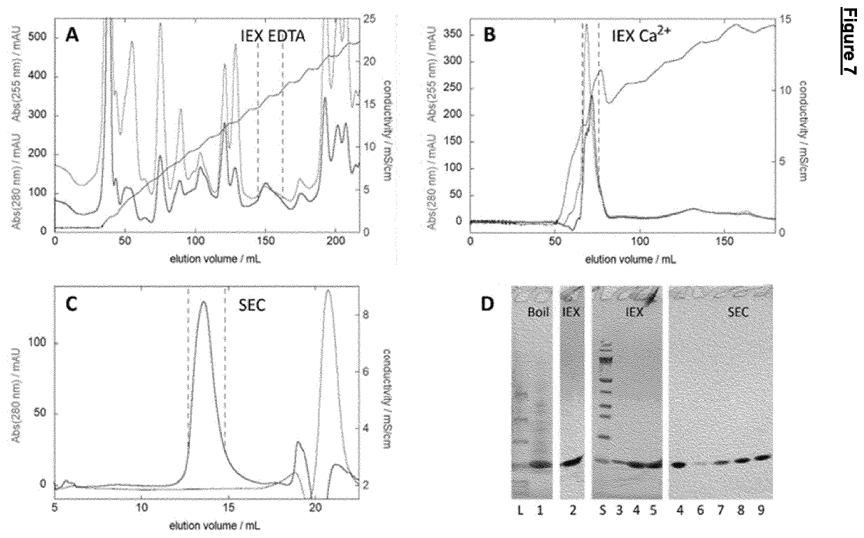

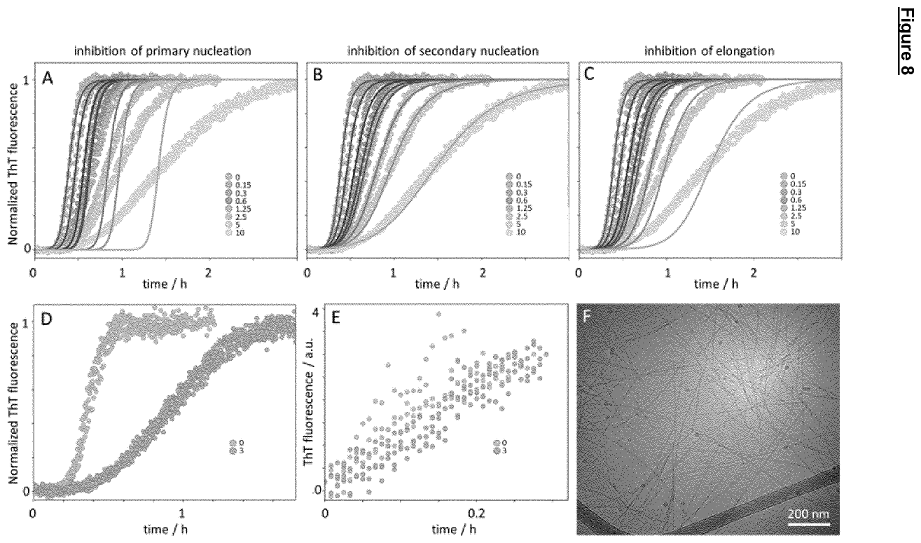

The eluates after three rounds of selection were subjected to PCR using primers inside the constant region of S100G followed by NGS to provide information on the retrieved side and loops sequences. The frequency of the most abundant sequence and the number of sequences that occurred at least 100 times after three rounds of selection are listed in Table 4. The frequency of occurrence of the top 500 hits in each case is displayed in FIG. 5, and the sequences of the top 10 hits in each case are listed in Table 5. The sequences of two investigated hits with alignment clusters of similar sequences are shown in FIG. 6.

TABLE 4
Summary of the eight screens for Aβ binders. The
highest frequency of occurrence of any sequence (Top
freq) and the number of sequences found at least 100
times (N seq) in the NGS data after three rounds of selection.

| | | | | Top | |
|---|---|---|---|---|---|
| Selection | Library | Target | Depleted on | freq | N seq |
| Loop40mono | S100GLoop | Aβ40 | — | 1.5% | 417 |
| | | monomer | | | |
| Loop40fib | S100GLoop | Aβ40 fibril | Aβ40 | 1.4% | 6212 |
| | | | monomer | | |
| Loop42mono | S100GLoop | Aβ42 | — | 7.2% | 661 |
| | | monomer | | | |
| Loop42fib | S100GLoop | Aβ42 fibril | Aβ42 | 1.7% | 3082 |
| | | | monomer | | |
| Side40mono | S100GSide | Aβ40 | — | 3.7% | 458 |
| | | monomer | | | |
| Side40fib | S100GSide | Aβ40 fibril | Aβ40 | 2.1% | 2483 |
| | | | monomer | | |
| Side42mono | S100GSide | Aβ42 | — | 0.9% | 3874 |
| | | monomer | | | |
| Side42fib | S100GSide | Aβ42 fibril | Aβ42 | 8.5% | 690 |
| | | | monomer | | |

TABLE 5
The 10 most frequent sequences obtained in each selection.

| | | | | | | | |
|---|---|---|---|---|---|---|---|
| | SEQ | | SEQ ID | | SEQ | | SEQ ID |
| Aβ40 mono | ID NO: | Aβ40 fibril | NO: | Aβ42 mono | ID NO: | Aβ42 fibril | NO: |

Side library

| | | | | | | | |
|---|---|---|---|---|---|---|---|
| VI--VI--DD | 97 | DQ--EG--HP | 107 | TA--RN--WA | 117 | WT--TV--VW | 127 |
| AP--LH--DE | 98 | SL--FP--DD | 108 | TH--PI--LS | 118 | PT--QI--HW | 128 |
| HK--SQ--WF | 99 | DA--NK--NP | 109 | MA--HA--GY | 119 | TP--AI--SY | 129 |
| PK--NR--FG | 100 | AY--PY--IP | 110 | MR--VE--HV | 120 | PP--QN--HF | 130 |
| YG--GY--HG | 101 | IW--NI--EL | 111 | NE--EH--NE | 121 | FL--DV--HA | 13 |
| EH--TL--LF | 102 | AI--YA--FK | 112 | GP--YA--IF | 122 | YY--TE--YV | 132 |
| YM--FI--AI | 103 | DY--PN--PP | 113 | HN--PD--QL | 123 | RQ--GF--LF | 133 |
| SI--RH--YE | 104 | EF--GP--EA | 114 | HS--EL--EY | 124 | KN--YW--VE | 134 |
| GD--HK--LY | 105 | GN--MQ--MM | 115 | VM--AN--GT | 125 | DS--KY--YW | 135 |
| FW--NI--VY | 106 | YY--DD--MP | 116 | HF--SR--NY | 126 | DD--FM--RM | 136 |

Loop library

| | | | | | | | |
|---|---|---|---|---|---|---|---|
| VNIGLEY | 53 | IRQDAQA | 63 | QGKSVPA | 73 | EGVNEFF | 83 |
| LFVMTRM | 54 | RHRKPFE | 64 | YLTIRLM | 74 | IRWTVMM | 84 |
| HHYTVFM | 55 | GLDTRHD | 65 | ASNTYFS | 75 | GYRWWW | 85 |
| ILAIFFV | 56 | TLGKMHH | 66 | LIWGFKT | 76 | DRSNSPE | 86 |
| EDHREMD | 57 | KNMQMWV | 67 | PDPLDFD | 77 | RAHDASI | 87 |
| HPRSTAV | 58 | DYQPQGI | 68 | DLVSFYY | 78 | VHTKAAA | 88 |
| IMGYPLN | 59 | WPVGHAT | 69 | SWMAILV | 79 | DQWIEHV | 89 |
| FGVHEWV | 60 | MKQGPVY | 70 | SSWRGTT | 80 | NEMFVVW | 90 |
| PTDIWAW | 61 | FKRSWIF | 71 | HHQMTKS | 81 | DRQWYPA | 91 |
| PIHESEH | 62 | HITHNET | 72 | PWTVPVD | 82 | VHKFGHI | 92 |

### Production of Candidate SXkmers

The loop sequence GGGYLTIRLMGGG (SEQ ID NO: 138) and the side patch VI-WI-DD (SEQ ID NO: 137) were selected based on the frequency of occurrence and the presence of many homologous sequences for both (FIG. 6). These were reintroduced into human S100G and expressed as free proteins, SXkmers. The proteins were found to be expressed at a very high yield of ca. 1 g/L (SXkmer-YLTIRLM) and 200 mg/L (SXkmer-VI-WI-DD) and were purified using sonication, boiling, two anion exchange steps in EDTA and Ca2+, respectively, and size exclusion chromatography (Methods & FIG. 7). The full amino acid sequences of these two proteins are shown in FIG. 6.

### Characterisation of Labelled Peptides by Size Exclusion Chromatography

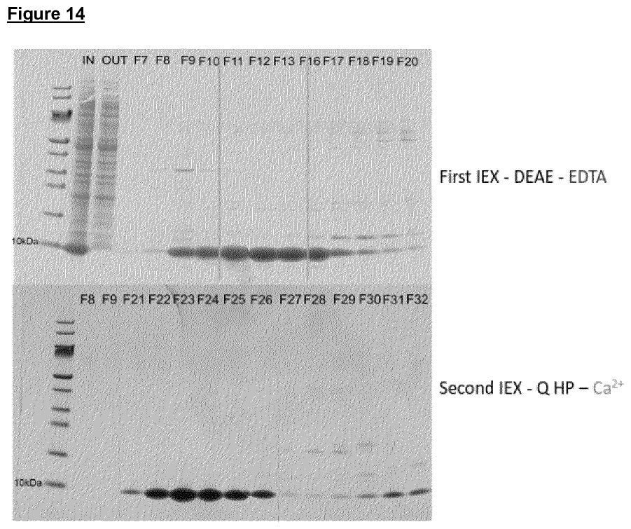

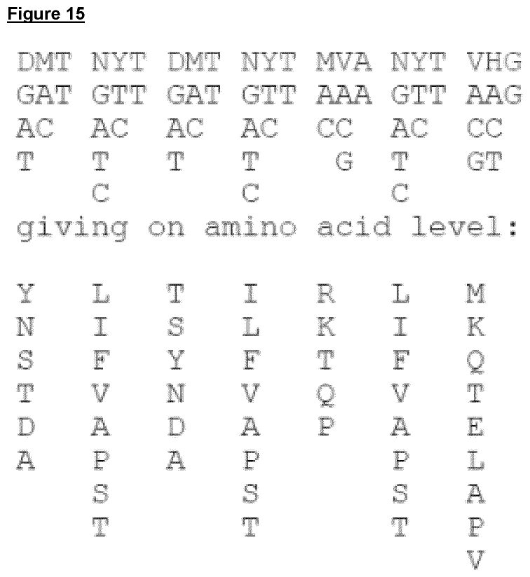

Peptides were purified using a twin ion exchange (IEX) approach in microwell plates (FIG. 14) and subsequently labelled with AlexaFlour labels. As shown in FIG. 14 it is possible to make a peptide composition at a very high concentration up to 1 g/L. The twin IEX approach leads to a two-fold protein purification. In the first purification there is no Ca⁺ present which ensured that the protein samples that were less negatively charged were selected to go forward to the second IEX purification (e.g. select the samples between located between the bars noted on the top panel of FIG. 14). The second stage IEX uses a Cat buffer which is more positive than the first IEX step. Samples were selected within the bars noted on the bottom panel of FIG. 14. By carrying out the second IEX purification highly purified protein samples were produced.

Aggregation kinetics—Aβ42

The effect of SXkmer-YLTIRLM on the aggregation kinetics of Aβ42 was monitored using thioflavin T (ThT) fluorescence (FIG. 8). The data were analysed using the Amylofit platform (Meisl 2016) and three modes of fitting were tested assuming selective reduction of the rate constant for primary nucleation (kₙ), secondary nucleation (k₂) and elongation (k+). Clearly, the model assuming selective reduction of k₂ fits the data best, indicating that YLTIRLM selectively inhibits secondary nucleation. The results of seeded experiments show that SXkmer-YLTIRLM clearly inhibits the aggregation starting from monomers supplemented with a low concentration (1%) of seed fibrils, implying blocking of secondary nucleation, but has little effect on heavily seeded reactions (30% seed) implying that SXkmer-YLTIRLM does not interfere with elongation of the seed fibrils (FIG. 8).

### Aggregation Kinetics—Aβ40

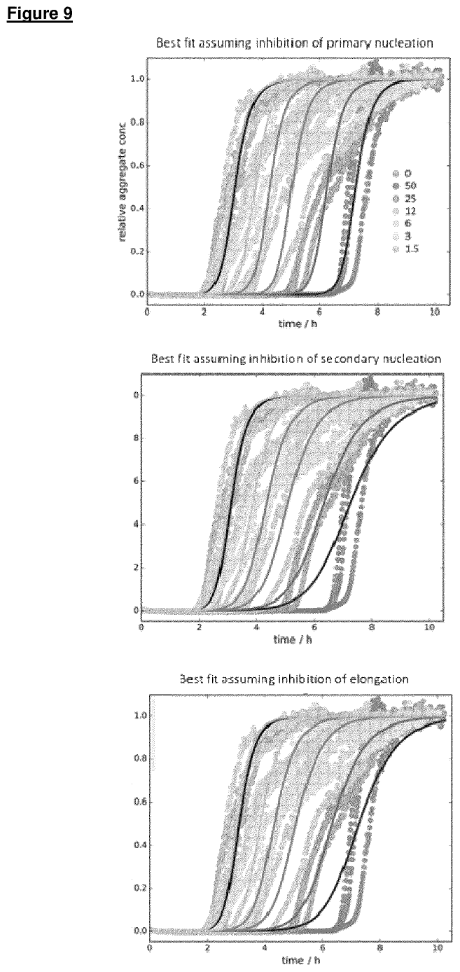

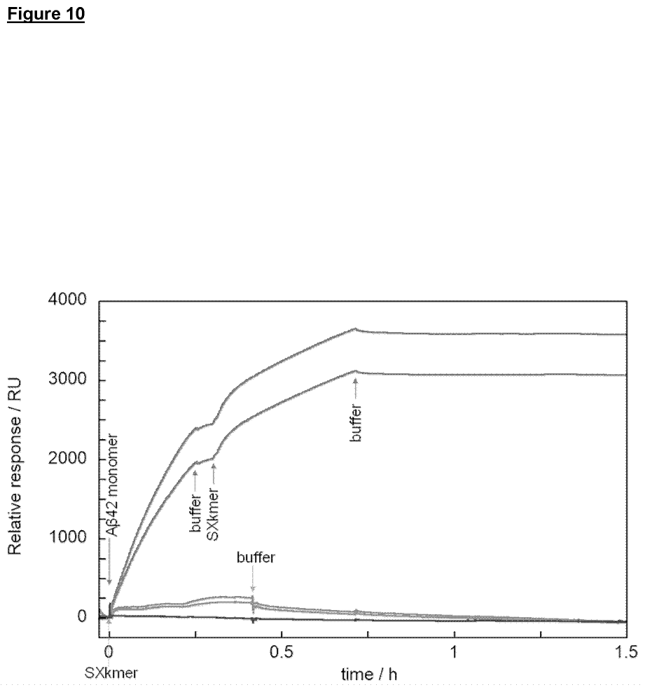

The effect of SXkmer-VI-WI-DD, derived from the side library on the Aβ40 aggregation kinetics was monitored by thioflavin T (ThT) fluorescence (FIG. 9). The data were analysed using the Amylofit platform (Meisl 2016). Three modes of fitting were tested assuming selective reduction of the rate constant for primary nucleation (kₙ), secondary nucleation (k₂) and elongation (k₊). Here, the distinction between models is less clear, but the model assuming selective reduction of kₙ fits the data best, suggesting that SXkmer-VI-WI-DD inhibits primary nucleation. This was confirmed using seeded aggregation reactions, bypassing primary nucleation, in which case no effect of SXkmer-VI-WI-DD was observed (FIG. 9).

### Binding Analyses by Surface Plasmon Resonance

The interaction between the SXkmer-YLTIRLM and Aβ42 was monitored by surface plasmon resonance (SPR, FIG. 10) using sensorchip surfaces with immobilized monomer or fibrils. Little or no binding was observed to flow cells with immobilized Aβ42 monomers, and only small changes in flow cells with immobilized and equilibrated Aβ42 fibrils. In contrast, a very significant increase in response was observed if Aβ42 monomers were injected over immobilized Aβ42 fibrils just prior to the injection of SXkmer-YLTIRLM (FIG. 10). While some of the injected monomers elongate the immobilized fibrils, a large fraction of the injected monomers adsorbs on the sides of fibrils leading to the formation of fibril-associated oligomers (Cohen 2018). These oligomers may detach or convert to fibrillar structure and detach (Michaels 2020), and our data imply that if SXkmer-YLTIRLM is injected while most of these oligomers remain on the fibrils, there is a strong interaction between SXkmer-YLTIRLM and the oligomers on the fibrils.

### Binding Analyses in Solution by FRET and MDS

The interaction between the SXkmer-YLTIRLM and Aβ42, labeled with Alexa488 and Alexa555, respectively, was monitored using FRET (FIG. 12), in which case an interaction between SXkmerYLTIRLM and Aβ42 would result in energy transfer between Alexa488 (emission at 522 nm) and Alexa555 and therefore reduced fluorescence emission at 522 nm. As shown in FIG. 12B, the fluorescence spectra recorded in the presence of monomers (2 min) or end-stage fibrils (7 h) are closely similar to the sum of spectra recorded separately for the two components, thus not revealing any interaction with monomers or fibrils. However, when SXkmer-YLTIRLM-Alexa488 was added to samples collected during an Aβ42 aggregation process (FIG. 12D), the fluorescence signal at 522 nm was attenuated, implying that SXkmer-YLTIRLM interacts with reaction intermediates rather than monomers or fibrils. The transfer efficiency, calculated from the signal intensity at 522 nm (FIG. 12C), displayed a sharp rise, a maximum around the midpoint of the fibril formation reaction, and a slower return to baseline. When instead fluorescence spectra were recorded continuously during an ongoing aggregation reaction in samples of 9 μMAβ4, 1 μM Aβ42-Alexa555, and 0.1 μM SXkmer-YLTIRLM-Alexa488, the FRET effect developed over time but did not disappear over the time scale of the experiment (FIG. 13), in line with the low dissociation rate inferred from the SPR data (FIG. 10).

The interaction between the SXkmer-YLTIRLM-Alexa647 and Aβ42 was also monitored using MDS. Samples of unlabeled Aβ42 were withdrawn at different time points from an ongoingreaction (FIG. 12D) and mixed with SXkmer-YLTIRLM-Alexa647, resulting in final total concentrations of 10 μMAβ42 and 1 nM SXkmer-YLTIRLM-Alexa647. An interaction between SXkmerYLTIRLM and Aβ42 would result in a reduced average diffusion rate and an increased apparent hydrodynamic radius (Rh) of Alexa647-labeled species. The apparent Rh versus the reaction time (FIG. 12C) indeed displayed a short lag phase, a sharp rise, a maximum close to the midpoint of the fibril formation reaction, and a slower return to baseline.

### Conclusions

Inhibitors of discrete steps along an amyloid formation process is a tractable goal. Here we identify an SXkmer protein that selectively inhibits secondary nucleation of Aβ42, and another one that seems to inhibit primary nucleation of Aβ40. These molecules represent a significant advancement in the goal of preventing and curing Alzheimer's Disease.

### Secondary Nucleation Inhibitors

Selective inhibition of secondary nucleation can arise by more than one mechanism (Arosio 2016; Michaels 2020). Proteins that bind along the sides of fibrils may block the catalytic sites for secondary nucleation. Molecules that bind to oligomeric structures formed on the fibril surface may prevent their conversion to fibrillar structure. Examples in the first category are the Brichos chaperone domain, which fully blocks secondary nucleation of Aβ42 (Cohen 2015) and the antibody Aducanumab that may reduce the rate of secondary nucleation of Aβ42 up to three-fold (Linse 2020). These two examples represent secondary nucleation inhibitors in the form of human-derived proteins. Brichos domains are typically found in proteins with highly amyloidogenic segments, for example lung surfactant protein C, where the Brichos domain protects against amyloid formation in the lung. Aducanumab was isolated from an old and cognitively normal individual with no sign of dementia and has been found to reduce brain amyloid and clinical symptoms (Sevigny 2016; Haeberlein 2020), and was recently approved by the US Food and Drug Administration (FDA) for AD treatment. An example in the latter category is DNAJB6, see below.

### Primary Nucleation Inhibitors

Selective inhibition of primary nucleation can also arise by more than one mechanism (Arosio 2016; Michaels 2020). Proteins that bind to monomer work through the law of mass action and reduce the primary nucleation rate by lowering the free concentration of monomers. Molecules that bind to oligomeric structures, or form co-oligomers with amyloid proteins, may prevent their conversion to fibrillar structure. The chaperone DNAJB6, which seems to inhibit both primary and secondary nucleation of Aβ42 (Månsson 2014 & 2018), is a chaperone protein that inhibits amyloid formation and increases the effective solubility of a range of aggregation-prone proteins (Månsson 2014b). It has been found to bind to oligomeric rather than monomeric forms of Aβ40 (Österlund 2020). DNAJB6 may thus interfere with oligomers both in solution and on the fibril surface and supress their nucleation into regular fibrillar structure (Månsson 2014 & 2018; Linse 2021) and may increase the effective solubility of the amyloid protein through the formation of co-aggregates of a different structure compared to the peptide-alone fibrils (Linse 2021). Naturally occurring small molecules such as curcumin and resveratrol have been shown to bind to the N-terminus (residues 5-20) of Aβ42 monomers thereby interfering with primary nucleation (Fu 2014).

### Novel Protein Inhibitors

The present study was designed with a goal of developing novel proteins in the form of primary or secondary nucleation inhibitors. Previous efforts in this direction have employed an “antibody scanning” strategy to generation of antibodies against different linear epitopes along the Aβ42 sequence (Aprile 2017) or phage display selection of Aβ42 fibril-specific binders from a library of single-chain variable fragments (scFvs, Munke 2017). The affibody XXX is an example of a monomer binder, which supresses primary nucleation through reduced monomer concentration. The latter approach has been extended and modified in two ways in the current work. Firstly, two novel SXkmer libraries, built on the highly inert and stable scaffold S100G, were screened for binders to enable derivation of more stable binders compared to scFvs. Secondly, we retrieved not only fibril-specific binders but oligomer-binding proteins.

### Oligomer Binders

A major challenge in the field is the derivation of oligomer-specific binders. Although SXkmer-YLTIRLM was found in the selection versus immobilized Aβ42 monomers, it seems to bind to oligomers rather than fibrils. Most likely the conditions used for the coupling of purified monomers to silica nanoparticles resulted in a mixture of immobilized monomers and oligomers. This means that in the monomer selection we may have retrieved both monomer binders and oligomer binders. SXkmer-YLTIRLM belongs to the latter category because the SPR analysis fails to detect any interaction with monomers or mature fibrils. However, if Aβ42 monomers are injected over Aβ42 fibrils right before the injection of SXkmer-YLTIRLM, a strong increase in mass on the surface was observed indicating an interaction between SXkmer-YLTIRLM and oligomeric structures on fibrils (FIG. 10).

### Reverse Engineering

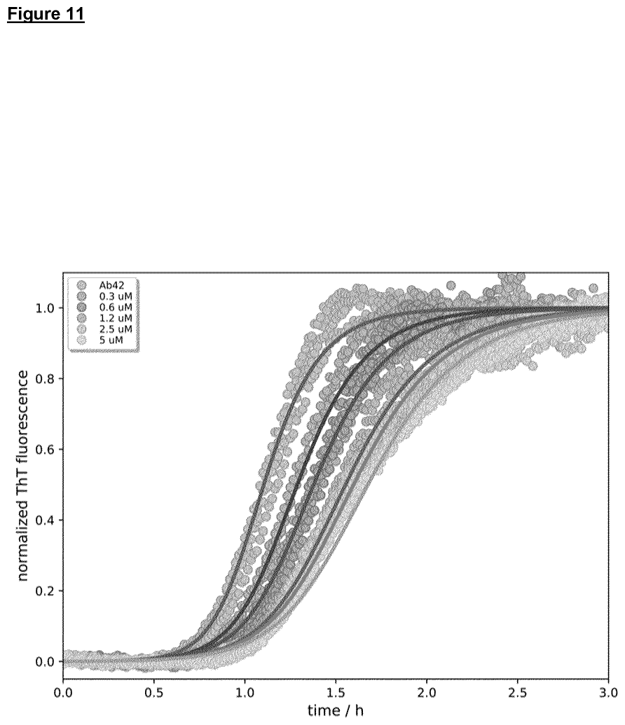

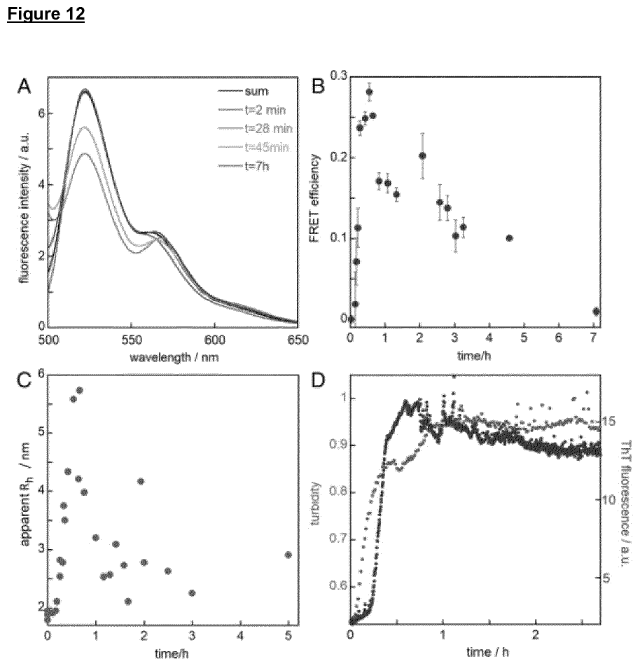

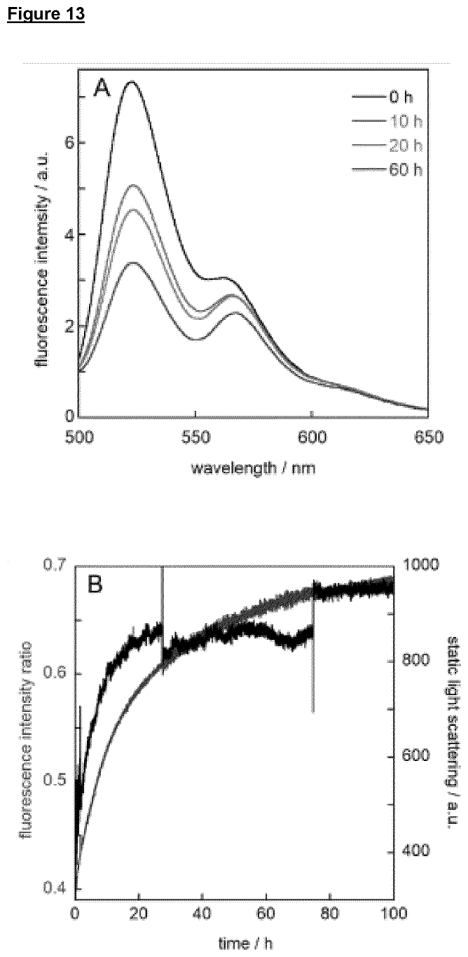

The current results may stimulate reverse engineering of a peptide based on the loop sequence of selected proteins from the SXkmer libraries as leads for future therapeutic modalities. Here we show that the inhibitory effect of SXkmer-YLTIRLM is retained by the synthetic linear peptide corresponding to the 7 amino acid Loop sequence (FIG. 11).

### Example 3—SXkmer Library Development and Testing

Following the work above a SXkmer library was developed based on the “YLTIRLM” SXkmer detailed in Examples 1 and 2. This library was devised using the same methods as detailed above in Example 1 but was based on the “YLTIRLM” SXkmer (FIG. 15).

This YLTIRLM library has a variable SXkmer sequence but is based on the YLTIRLM sequence and is therefore a more focused library than above. The SXkmer sequences produced by this library are shown below in Table 6.

TABLE 6
SXkmer amino acid sequences included in the new
library based on the YLTIRLM SXkmer. The
7-mers (SEQ ID NOs: 12-32) are focussed whereas
the 5-mers (SEQ ID NOs: 33-39) are new and a
naive library to Aβs.

| SXkmer | SEQ ID NO: |
|---|---|
| SFTFRAA | 12 |
| SLNTTFP | 13 |
| SSSLPFP | 14 |
| NASFRTP | 15 |
| NFAIPLL | 16 |
| SVTSPFT | 17 |
| SFNFTIA | 18 |
| SLNVTFV | 19 |
| AIDSPFV | 20 |
| YVYLQWV | 21 |
| YFSFRPL | 22 |
| SVYVRLV | 23 |
| SASFRLL | 24 |
| SVSIKVK | 25 |
| YLYVTLV | 26 |
| YLYLRIM | 27 |
| SFYVRFL | 28 |
| TLSIRVA | 29 |
| DVDLKPL | 30 |
| APSARLE | 31 |
| TVDTKLL | 32 |
| INLAF | 33 |
| VKGSA | 34 |
| RRFVN | 35 |
| SSTTS | 36 |
| SLTLT | 37 |
| RLYLT | 38 |
| VGSMF | 39 |

### Methods

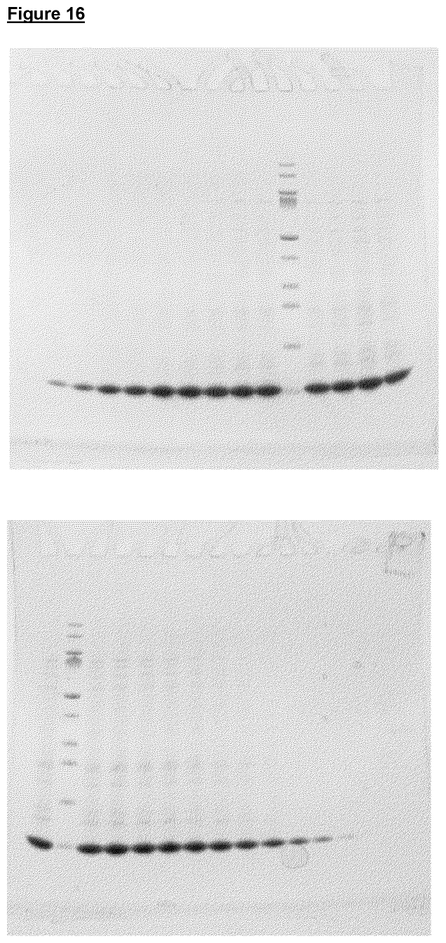

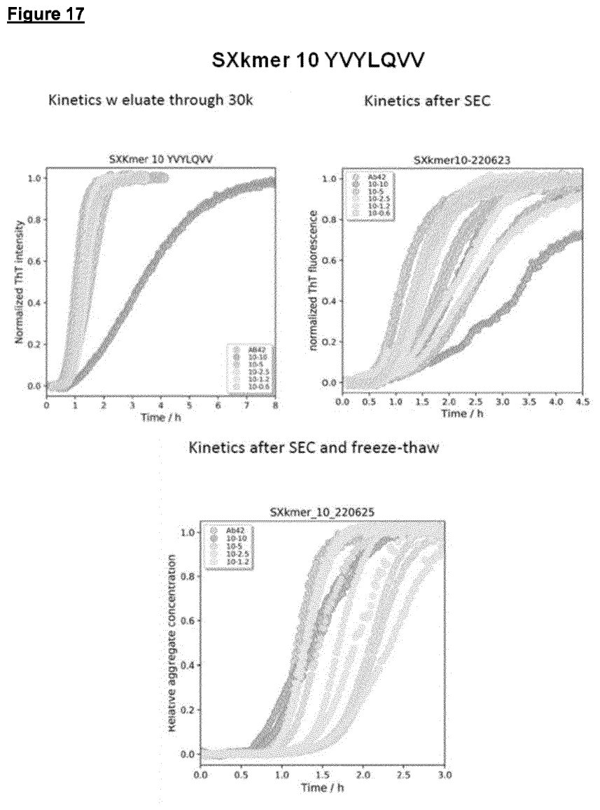

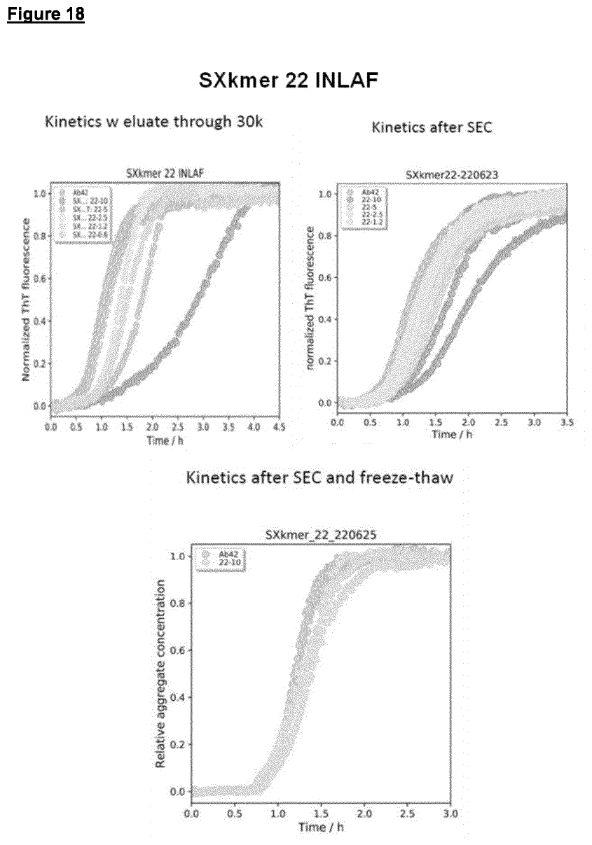

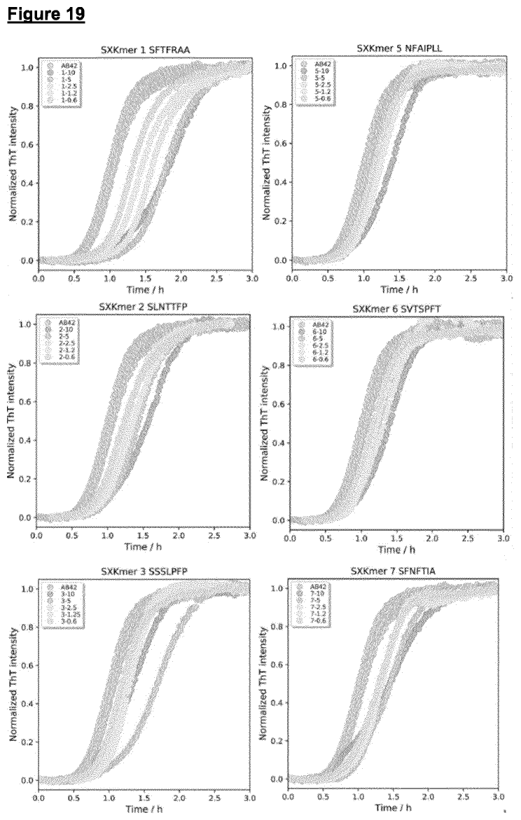

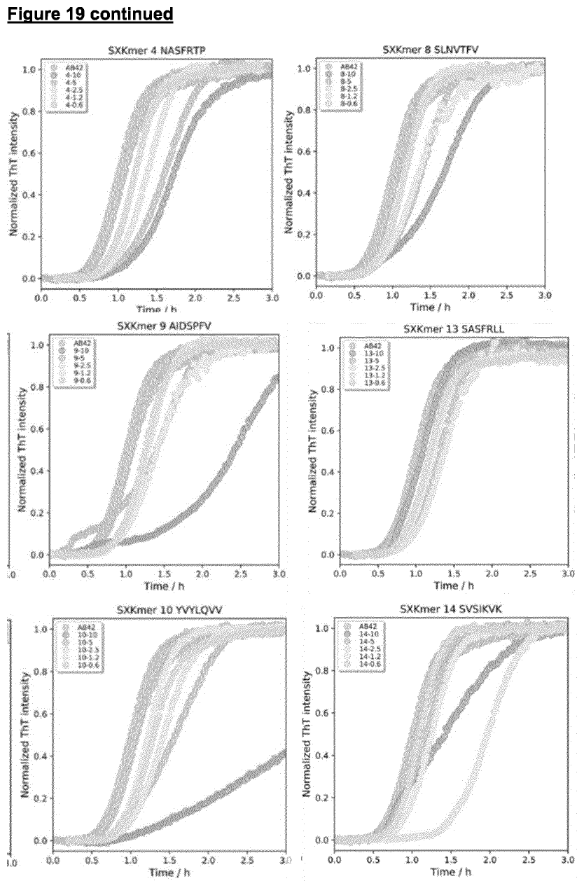

The new library was expressed using the same methods as Example 1 above and shown in FIG. 16.

The effect on Aβ Aggregation of exposure to the SXkmers from the new library was investigated using the same methods as detailed above in Examples 1 and 2.

### Results

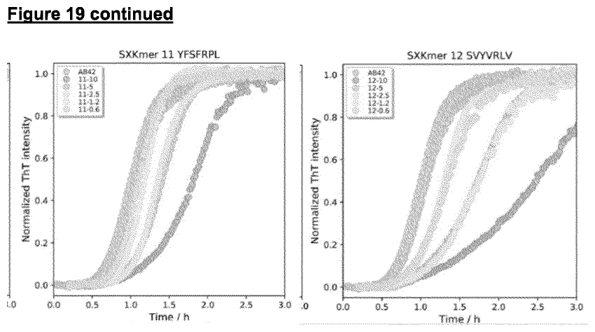

As shown in FIG. 17 the 7-mer SXkmer “YVYLQVV” (SEQ ID NO: 21) was tested for its ability to inhibit Aβ Aggregation either after elution of the SXkmer through 30k, after size exclusion chromatography (SEC) or SEC followed by a freeze-thaw cycle.

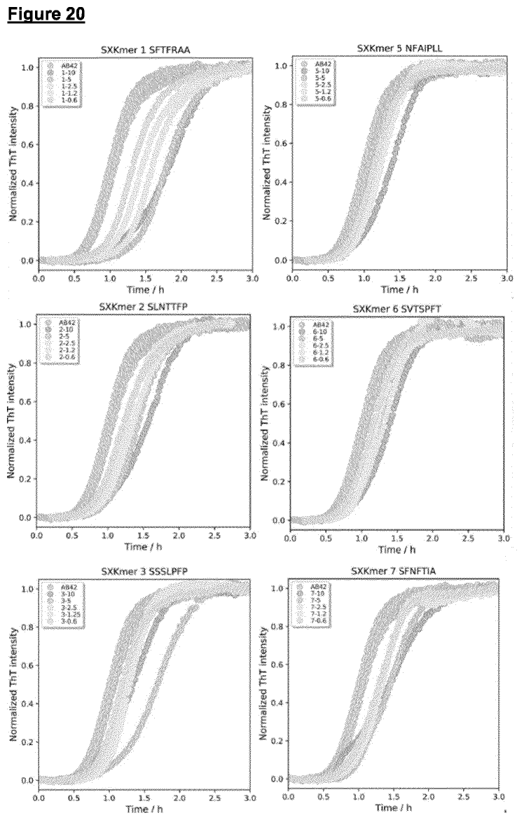

FIG. 17 shows that this 7-mer is able to inhibit Aβ Aggregation however this inhibition is much more effective after SEC and is only marginally affected after a freeze-thaw cycle. FIG. 18 shoes the same data for the 5-mer “INLAF” (SEQ ID NO: 33) SXkmer which demonstrates the same pattern as that of the 7-mer in FIG. 17.

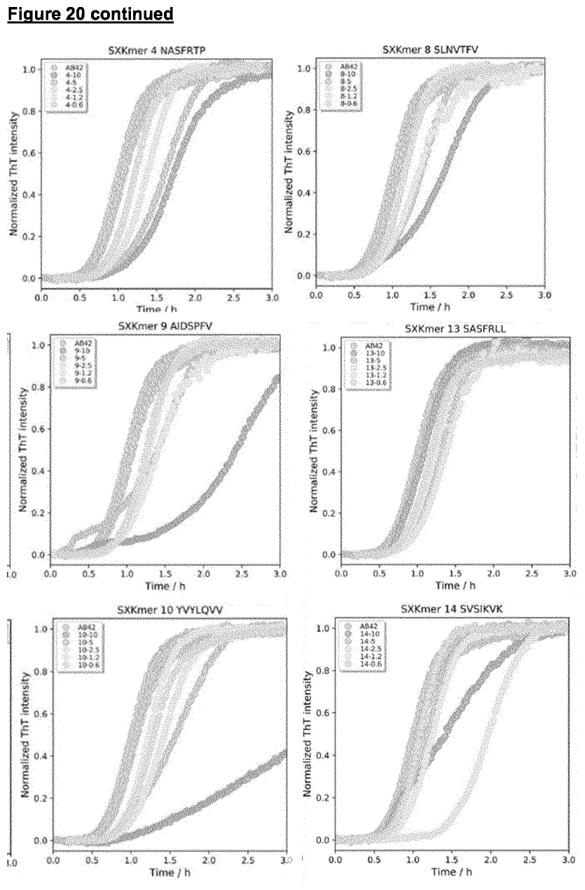

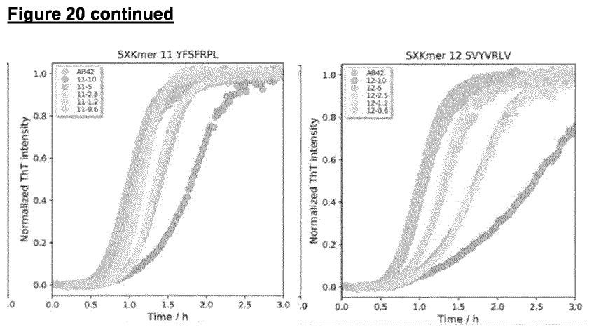

FIG. 19 shows the data for Aβ Aggregation inhibition by all the 7-mer SXkmers listed in Table 6 and FIG. 20 shows the data for Aβ Aggregation inhibition by all the 5-mer SXkmers listed in Table 6.

The forgoing embodiments are not intended to limit the scope of the protection afforded by the claims, but rather to describe examples of how the invention may be put into practice.

### Sequences not Listed Elsewhere in the Description:

TABLE 7
Sequences not listed elsewhere in the description. “N” may be any
DNA residue and “K” is residues G or T in SEQ ID NOs 40-44 above.
“D” is residues G, A, or T and “M” is residues A or C and “Y” is
residues T or C and “V” is residues A, C or G and “H” is residues A,
C or T in SEQ ID NO: 44 above.

| | |
|---|---|
| SEQ ID NO: 40 | Lib35: GCCATGGGTAAATCTCCGGAAGAACTGAAACGTATCTTCGAA |
| | AAATACGCTGCTAAAGAAGGTGACCCGGACCAGCTGTCTAAAGAC |
| | GAACTGAAACTGCTGATCCAGGCTGAATTCCCGTCTCTGCTGAAA |
| | GGTATGGGTGGCGGTNNKNNKNNKNNKNNKGGTGGTGGCTCTAC |
| | CCTGGACGACCTGTTCCAGGAACTGG |
| SEQ ID NO: 41 | Lib36: GCCATGGGTAAATCTCCGGAAGAACTGAAACGTATCTTCGA |
| | AAAATACGCTGCTAAAGAAGGTGACCCGGACCAGCTGTCTAAAGA |
| | CGAACTGAAACTGCTGATCCAGGCTGAATTCCCGTCTCTGCTGAA |
| | AGGTATGGGTGGCGGTNNKNNKNNKNNKNNKNNKGGTGGTGGCT |
| | CTACCCTGGACGACCTGTTCCAGGAACTGG |
| SEQ ID NO: 42 | Lib37: GCCATGGGTAAATCTCCGGAAGAACTGAAACGTATCTTCGA |
| | AAAATACGCTGCTAAAGAAGGTGACCCGGACCAGCTGTCTAAAG |
| | ACGAACTGAAACTGCTGATCCAGGCTGAATTCCCGTCTCTGCTG |
| | AAAGGTATGGGTGGCGGTNNKNNKNNKNNKNNKNNKNNKGGTG |
| | GTGGCTCTACCCTGGACGACCTGTTCCAGGAACTGG |
| SEQ ID NO: 43 | Lib17: GCCATGGGTAAATCTCCGGAAGAACTGAAACGTATCTTCGAA |
| | AAATACGCTGCTAAAGAAGGTGACCCGGACCAGCTGTCTAAAGA |
| | CGAACTGAAACTGCTGATCCAGGCTGAATTCCCGTCTCTGCTGA |
| | AAGGTATGGGTNNKNNKNNKNNKNNKNNKNNKGGTTCTACCCTG |
| | GACGACCTGTTCCAGGAACTGG |
| SEQ ID NO: 44 | YLTLib: GCCATGGGTAAATCTCCGGAAGAACTGAAACGTATCTTCG |
| | AAAAATACGCTGCTAAAGAAGGTGACCCGGACCAGCTGTCTAAAG |
| | ACGAACTGAAACTGCTGATCCAGGCTGAATTCCCGTCTCTGCTGA |
| | AAGGTATGGGTGGCGGTDMTNYTDMTNYTMVANYTVHGGGTGGT |
| | GGCTCTACCCTGGACGACCTGTTCCAGGAACTGG |

### REFERENCES

Hebert, L. E.; Weuve, J.; Scherr, P. A.; Evans, D. A. Alzheimer disease in the United States (2010-2050) estimated using the 2010 census. Neurology 2013, 80, 1778-1783.
Huang M, Li Z, Li D, Walker S, Greenan C, Kennedy R. Structure-based design of HSPA5 inhibitors: from peptide to small molecule inhibitors. Bioorg Med Chem Lett. 2013 May 15; 23 (10): 3044-50. doi: 10.1016/j.bmcl.2013.03.035.
Selkoe, D. J. The therapeutics of Alzheimer's disease: where we stand and where we are heading. Ann. Neurol. 2013, 74, 328-336.
Hardy J A, Higgins G A. Alzheimer's disease: the amyloid cascade hypothesis. Science. 1992 Apr. 10; 256 (5054): 184-5.
Walsh D M, Klyubin I, Fadeeva J V, Cullen W K, Anwyl R, Wolfe M S, Rowan M J, Selkoe D J. Naturally secreted oligomers of amyloid beta protein potently inhibit hippocampal long-term potentiation in vivo. Nature. 2002 Apr. 4; 416 (6880): 535-9.
Colvin M T, Silvers R, Ni Q Z, Can T V, Sergeyev I, Rosay M, Donovan K J, Michael B, Wall J, Linse S, Griffin R G. Atomic Resolution Structure of Monomorphic Aβ42 Amyloid Fibrils. J Am Chem Soc. 2016 Aug. 3; 138 (30): 9663-74.
Wälti M A, Ravotti F, Arai H, Glabe C G, Wall J S, Böckmann A, Güntert P, Meier B H, Riek R. Atomic-resolution structure of a disease-relevant Aβ (1-42) amyloid fibril. Proc Natl Acad Sci USA. 2016 Aug. 23; 113 (34): E4976-84.
Schütz A K, Hornemann S, Wälti M A, Greuter L, Tiberi C, Cadalbert R, Gantner M, Riek R, Hammarstrom P, Nilsson K P R, Bockmann A, Aguzzi A, Meier B H. Binding of Polythiophenes to Amyloids: Structural Mapping of the Pharmacophore. ACS Chem Neurosci. 2018 Mar. 21; 9 (3): 475-481.
Fitzpatrick A W P, Falcon B, He S, Murzin A G, Murshudov G, Garringer H J, Crowther R A, Ghetti B, Goedert M, Scheres S H W. Cryo-EM structures of tau filaments from Alzheimer's disease. Nature. 2017 Jul. 13; 547 (7662): 185-190.
J. Sevigny et al., The antibody aducanumab reduces Aβ plaques in Alzheimer's disease. Nature 537, 50-56 (2016).
S. B. Haeberlein et al., Emerge and Engage topline results: Phase 3 studies of aducanumab in early Alzheimer's disease. Alzheimer's & Dementia 16 (2020).
Fu Z. et al Biochemistry 2014, 53, 50, 7893-7903.
Mei et al., ACS Chem Neurosci 2021.
Cohen S I, Linse S, Luheshi L M, Hellstrand E, White D A, Rajah L, Otzen D E, Vendruscolo M, Dobson C M, Knowles T P. Proliferation of amyloid-β42 aggregates occurs through a secondary nucleation mechanism. Proc Natl Acad Sci USA. 2013 Jun. 11; 110 (24): 9758-63.
Cohen S I A, Arosio P, Presto J, Kurudenkandy F R, Biverstal H, Dolfe L, Dunning C, Yang X, Frohm B, Vendruscolo M, Johansson J, Dobson C M, Fisahn A, Knowles T P J, Linse S. A molecular chaperone breaks the catalytic cycle that generates toxic Aβ oligomers. Nat Struct Mol Biol. 2015 March; 22 (3): 207-213.
Cohen S I A, Cukalevski R, Michaels T C T, Šarić A, Tornquist M, Vendruscolo M, Dobson C M, Buell A K, Knowles T P J, Linse S. Distinct thermodynamic signatures of oligomer generation in the aggregation of the amyloid-β peptide. Nat Chem. 2018 May; 10 (5): 523-531.
Aprile F A, Sormanni P, Perni M, Arosio P, Linse S, Knowles T P J, Dobson C M, Vendruscolo M. Selective targeting of primary and secondary nucleation pathways in Aβ42 aggregation using a rational antibody scanning method. Sci Adv. 2017 Jun. 21; 3 (6): e1700488.
Habchi J, Arosio P, Perni M, Costa A R, Yagi-Utsumi M, Joshi P, Chia S, Cohen S I, Müller M B, Linse S, Nollen E A, Dobson C M, Knowles T P, Vendruscolo M. An anticancer drug suppresses the primary nucleation reaction that initiates the production of the toxic Abeta42 aggregates linked with Alzheimer's disease. Sci Adv. 2016 Feb. 12; 2 (2): e1501244.
Habchi J, Chia S, Limbocker R, Mannini B, Ahn M, Perni M, Hansson O, Arosio P, Kumita J R, Challa P K, Cohen S I, Linse S, Dobson C M, Knowles T P, Vendruscolo M. Systematic development of small molecules to inhibit specific microscopic steps of Abeta42 aggregation in Alzheimer's disease. Proc Natl Acad Sci USA. 2017 Jan. 10; 114 (2): E200-E208.
Michaels T C T, Šarić A, Curk S, Bernfur K, Arosio P, Meisl G, Dear A J, Cohen S I A, Dobson C M, Vendruscolo M, Linse S, Knowles T P J. Dynamics of oligomer populations formed during the aggregation of Alzheimer's Abeta42 peptide. Nat Chem. 2020 May; 12 (5): 445-451.
Arosio P, Michaels T C, Linse S, Månsson C, Emanuelsson C, Presto J, Johansson J, Vendruscolo M, Dobson C M, Knowles T P. Kinetic analysis reveals the diversity of microscopic mechanisms through which molecular chaperones suppress amyloid formation. Nat Commun. 2016 Mar. 24; 7:10948.
Munke A, Persson J, Weiffert T, De Genst E, Meisl G, Arosio P, Carnerup A, Dobson C M, Vendruscolo M, Knowles T P J, Linse S. Phage display and kinetic selection of antibodies that specifically inhibit amyloid self-replication. Proc Natl Acad Sci USA. 2017 Jun. 20; 114 (25): 6444-6449.
Linse S, Scheidt T, Bernfur K, Vendruscolo M, Dobson C M, Cohen S I A, Sileikis E, Lundqvist M, Qian F, O'Malley T, Bussiere T, Weinreb P H, Xu C K, Meisl G, Devenish S R A, Knowles T P J, Hansson O. Kinetic fingerprints differentiate the mechanisms of action of anti-Aβ antibodies Nat Struct Mol Biol. 2020 December; 27 (12): 1125-1133.
Michaels T C T, Šarić A, Meisl G, Heller G T, Curk S, Arosio P, Linse S, Dobson C M, Vendruscolo M, Knowles T P J. Thermodynamic and kinetic design principles for amyloid-aggregation inhibitors. Proc Natl Acad Sci USA. 2020 Sep. 29; 117 (39): 24251-24257.
Månsson C, Arosio P, Hussein R, Kampinga H H, Hashem R M, Boelens W C, Dobson C M, Knowles T P, Linse S, Emanuelsson C. Interaction of the molecular chaperone DNAJB6 with growing amyloid-beta 42 (Aβ42) aggregates leads to sub-stoichiometric inhibition of amyloid formation. J Biol Chem. 2014a November 7; 289 (45): 31066-76.
Månsson C, Kakkar V, Monsellier E, Sourigues Y, Härmark J, Kampinga H H, Melki R, Emanuelsson C. DNAJB6 is a peptide-binding chaperone which can suppress amyloid fibrillation of polyglutamine peptides at substoichiometric molar ratios. Cell Stress Chaperones. 2014b March; 19 (2): 227-39.
Månsson C, van Cruchten R T P, Weininger U, Yang X, Cukalevski R, Arosio P, Dobson C M, Knowles T, Akke M, Linse S, Emanuelsson C. Conserved S/T Residues of the Human Chaperone DNAJB6 Are Required for Effective Inhibition of Aβ42 Amyloid Fibril Formation. Biochemistry. 2018 Aug. 14; 57 (32): 4891-4902.
Linse, S., Sormanni, P., O'Connell, D. J., 2022. An aggregation inhibitor specific to oligomeric intermediates of Aβ42 derived from phage display libraries of stable, small proteins. Proceedings of the National Academy of Sciences 119 . . . doi: 10.1073/pnas.2121966119

1. A double EF-hand protein comprising the amino acid sequence of any one of SEQ ID NOs: 2-11 or an amino acid sequence having at least 50% sequence similarity or identity thereto, wherein X is any amino acid residue, wherein the sequence similarity or identity is determined based on the amino acid residues of the constant region of SEQ ID NOs: 2-11, and wherein SEQ ID NOs: 7-11 include an insert selected from the following: GGGXXXXXXXGGG, GGGXXXXXXGGG, GGGXXXXXGGG, GGGXXXXGGG, GGGXXXGGG, GGXXXXXXXGG, GGXXXXXXGG, GGXXXXXGG, GGXXXXGG, GGXXXGG, GXXXXXXXG, GXXXXXXG, GXXXXXG, GXXXXG, GXXXG, XXXXXXX, XXXXXX, XXXXX, XXXX and XXX.

2. The protein of claim 1, wherein the sequence similarity or identity is at least 80%.

3. The protein of claim 2, wherein the sequence similarity or identity is at least 90%.

4. The protein of any one of claims 1-3, wherein X is any amino acid residue other than cysteine (C).

5. The protein of claim 4, wherein X is an amino acid residue selected from: alanine (A), arginine (R), asparagine (N), aspartic acid (D), glutamine (Q), glutamic acid (E), glycine (G), histidine (H), isoleucine (I), leucine (L), lysine (K), methionine (M), phenylalanine (F), proline (P), serine(S), threonine (T), tryptophan (W), tyrosine (Y) or valine (V).

6. The protein of any one of claims 1-5, wherein the protein is non-wild-type.

7. A nucleic acid encoding the protein of any one of claims 1-6.

8. A cell or virus particle comprising the nucleic acid of claim 7.

9. A library comprising two or more double EF-hand proteins of different amino acid sequences.

10. The library of claim 9, wherein the amino acid sequences of the double EF-hand proteins differ in at least 3 variable positions.

11. The library of claim 10, wherein the variable positions are located within an EF-hand or between two EF-hands.

12. The library of any one of claims 9-11, wherein the double EF-hand proteins comprise the amino acid sequence of any one of SEQ ID NOs: 2-11 or an amino acid sequence having at least 50% sequence similarity or identity thereto, wherein X is any amino acid residue, wherein the sequence similarity or identity is determined based on the amino acid residues of the constant region of SEQ ID NOs: 2-11, and wherein SEQ ID NOs: 7-11 include an insert selected from the following: GGGXXXXXXXGGG, GGGXXXXXXGGG, GGGXXXXXGGG, GGGXXXXGGG, GGGXXXGGG, GGXXXXXXXGG, GGXXXXXXGG, GGXXXXXGG, GGXXXXGG, GGXXXGG, GXXXXXXXG, GXXXXXXG, GXXXXXG, GXXXXG, GXXXG, XXXXXXX, XXXXXX, XXXXX, XXXX and XXX.

13. The library of any one of claims 9-12, wherein the proteins are phage-displayed.

14. A method of producing or expanding a library, the method comprising the step of collecting:
a) two or more double EF-hand proteins of different amino acid sequences; or
b) nucleic acids encoding such proteins.

15. A method of screening, the method comprising:
(a) exposing a target molecule to the proteins of the library of any one of claims 9-13; and
(b) assessing the binding affinity between the target molecule and one or more of the proteins.

16. A pharmaceutical composition comprising the protein of any one of claims 1-6 or the nucleic acid of claim 7.

17. The pharmaceutical composition of claim 16, wherein the protein comprises the amino acid sequence of any one of SEQ ID NOs: 2-6 wherein the variable positions are occupied by VI-WI-DD or the amino acid sequence of any one of SEQ ID NOs: 7-11 with an insert selected from the following: GGGYLTIRLMGGG, GGYLTIRLMGG, GYLTIRLMG and YLTIRLM, or an amino acid sequence having at least 50% sequence similarity or identity thereto wherein the sequence similarity or identity is determined based on the amino acid residues of the constant region of SEQ ID NOs: 2-11.

18. The pharmaceutical composition of claim 16 or 17 for use in the prevention or treatment of a medical condition or disease.

19. A method for the prevention or treatment of a medical condition or disease, the method comprising the administration of the pharmaceutical composition of claim 16 or 17 to a subject.

20. The use of the protein of any one of claims 1-6 in the manufacture of a medicament for the prevention or treatment of a medical condition or disease.

21. The use of claim 20, wherein the protein comprises the amino acid sequence of any one of SEQ ID NOs: 2-6 wherein the variable positions are occupied by VI-WI-DD or the amino acid sequence of any one of SEQ ID NOs: 7-11 with an insert selected from the following: GGGYLTIRLMGGG, GGYLTIRLMGG, GYLTIRLMG and YLTIRLM, or an amino acid sequence having at least 50% sequence similarity or identity thereto wherein the sequence similarity or identity is determined based on the amino acid residues of the constant region of SEQ ID NOS: 2-11.

22. The composition of claim 18, the method of claim 19 or the use of claim 20 or 21, wherein the medical condition or disease is cancer or a neurodegenerative disease.

23. The composition, method or use of claim 22, wherein the neurodegenerative disease is associated with the aggregation of Aβ.

24. The composition, method or use of claim 23, wherein the neurodegenerative disease is Alzheimer's disease.

25. A protein or peptide comprising or consisting of the amino acid sequence YLTIRLM for use in the prevention or treatment of a neurodegenerative disease.

26. The protein or peptide of claim 25, wherein the neurodegenerative disease is associated with the aggregation of Aβ.

27. The protein or peptide of claim 26, wherein the neurodegenerative disease is Alzheimer's disease.


SEQUENCE LISTING (138 sequences)

SEQ ID NO: 1 - protein, 79 aa, synthetic construct
  REGION 1..79: note = Wild-Type Human S100G
MSTKKSPEELKRIFEKYAAKEGDPDQLSKDELKLLIQAEFPSLLKGPNTLDDLFQELDKNGDGEVSFEEFQVLVKKISQ

SEQ ID NO: 2 - protein, 79 aa, synthetic construct
  REGION 1..79: note = Side library base template
  VARIANT 29..30: note = misc_feature - X can be any naturally occurring amino acid
  VARIANT 33..34: note = misc_feature - X can be any naturally occurring amino acid
  VARIANT 37..38: note = misc_feature - X can be any naturally occurring amino acid
MSTKKSPEELKRIFEKYAAKEGDPDQLSXXELXXLIXXEFPSLLKGPNTLDDLFQELDKNGDGEVSFEEFQVLVKKISQ

SEQ ID NO: 3 - protein, 78 aa, synthetic construct
  REGION 1..78: note = Side library base template
  VARIANT 28..29: note = misc_feature - X can be any naturally occurring amino acid
  VARIANT 32..33: note = misc_feature - X can be any naturally occurring amino acid
  VARIANT 36..37: note = misc_feature - X can be any naturally occurring amino acid
MTKKSPEELKRIFEKYAAKEGDPDQLSXXELXXLIXXEFPSLLKGPNTLDDLFQELDKNGDGEVSFEEFQVLVKKISQ

SEQ ID NO: 4 - protein, 77 aa, synthetic construct
  REGION 1..77: note = Side library base template
  VARIANT 27..28: note = misc_feature - X can be any naturally occurring amino acid
  VARIANT 31..32: note = misc_feature - X can be any naturally occurring amino acid
  VARIANT 35..36: note = misc_feature - X can be any naturally occurring amino acid
MKKSPEELKRIFEKYAAKEGDPDQLSXXELXXLIXXEFPSLLKGPNTLDDLFQELDKNGDGEVSFEEFQVLVKKISQ

SEQ ID NO: 5 - protein, 76 aa, synthetic construct
  REGION 1..76: note = Side library base template
  VARIANT 26..27: note = misc_feature - X can be any naturally occurring amino acid
  VARIANT 30..31: note = misc_feature - X can be any naturally occurring amino acid
  VARIANT 34..35: note = misc_feature - X can be any naturally occurring amino acid
MKSPEELKRIFEKYAAKEGDPDQLSXXELXXLIXXEFPSLLKGPNTLDDLFQELDKNGDGEVSFEEFQVLVKKISQ

SEQ ID NO: 6 - protein, 76 aa, synthetic construct
  REGION 1..76: note = Side library base template
  VARIANT 26..27: note = misc_feature - X can be any naturally occurring amino acid
  VARIANT 30..31: note = misc_feature - X can be any naturally occurring amino acid
  VARIANT 34..35: note = misc_feature - X can be any naturally occurring amino acid
MKSPEELKRIFEKYAAKEGDPDQLSXXELXXLIXXEFPSLLKGMSTLDDLFQELDKDGDGEVSFEEFQVLVKKISQ

SEQ ID NO: 7 - protein, 80 aa, synthetic construct
  REGION 1..80: note = Loop library base template
  VARIANT 48: note = misc_feature - X can be an insert containing one or more naturally occurring amino acids
MSTKKSPEELKRIFEKYAAKEGDPDQLSKDELKLLIQAEFPSLLKGPXNTLDDLFQELDKNGDGEVSFEEFQVLVKKISQ

SEQ ID NO: 8 - protein, 79 aa, synthetic construct
  REGION 1..79: note = Loop library base template
  VARIANT 47: note = misc_feature - X can be an insert containing one or more naturally occurring amino acids
MTKKSPEELKRIFEKYAAKEGDPDQLSKDELKLLIQAEFPSLLKGPXNTLDDLFQELDKNGDGEVSFEEFQVLVKKISQ

SEQ ID NO: 9 - protein, 78 aa, synthetic construct
  REGION 1..78: note = Loop library base template
  VARIANT 46: note = misc_feature - X can be an insert containing one or more naturally occurring amino acids
MKKSPEELKRIFEKYAAKEGDPDQLSKDELKLLIQAEFPSLLKGPXNTLDDLFQELDKNGDGEVSFEEFQVLVKKISQ

SEQ ID NO: 10 - protein, 77 aa, synthetic construct
  REGION 1..77: note = Loop library base template
  VARIANT 45: note = misc_feature - X can be an insert containing one or more naturally occurring amino acids
MKSPEELKRIFEKYAAKEGDPDQLSKDELKLLIQAEFPSLLKGPXNTLDDLFQELDKNGDGEVSFEEFQVLVKKISQ

SEQ ID NO: 11 - protein, 77 aa, synthetic construct
  REGION 1..77: note = Loop library base template
  VARIANT 45: note = misc_feature - X can be an insert containing one or more naturally occurring amino acids
MKSPEELKRIFEKYAAKEGDPDQLSKDELKLLIQAEFPSLLKGMXSTLDDLFQELDKDGDGEVSFEEFQVLVKKISQ

SEQ ID NO: 12 - protein, 7 aa, synthetic construct
SFTFRAA

SEQ ID NO: 13 - protein, 7 aa, synthetic construct
SLNTTFP

SEQ ID NO: 14 - protein, 7 aa, synthetic construct
SSSLPFP

SEQ ID NO: 15 - protein, 7 aa, synthetic construct
NASFRTP

SEQ ID NO: 16 - protein, 7 aa, synthetic construct
NFAIPLL

SEQ ID NO: 17 - protein, 7 aa, synthetic construct
SVTSPFT

SEQ ID NO: 18 - protein, 7 aa, synthetic construct
SFNFTIA

SEQ ID NO: 19 - protein, 7 aa, synthetic construct
SLNVTFV

SEQ ID NO: 20 - protein, 7 aa, synthetic construct
AIDSPFV

SEQ ID NO: 21 - protein, 7 aa, synthetic construct
YVYLQVV

SEQ ID NO: 22 - protein, 7 aa, synthetic construct
YFSFRPL

SEQ ID NO: 23 - protein, 7 aa, synthetic construct
SVYVRLV

SEQ ID NO: 24 - protein, 7 aa, synthetic construct
SASFRLL

SEQ ID NO: 25 - protein, 7 aa, synthetic construct
SVSIKVK

SEQ ID NO: 26 - protein, 7 aa, synthetic construct
YLYVTLV

SEQ ID NO: 27 - protein, 7 aa, synthetic construct
YLYLRIM

SEQ ID NO: 28 - protein, 7 aa, synthetic construct
SFYVRFL

SEQ ID NO: 29 - protein, 7 aa, synthetic construct
TLSIRVA

SEQ ID NO: 30 - protein, 7 aa, synthetic construct
DVDLKPL

SEQ ID NO: 31 - protein, 7 aa, synthetic construct
APSARLE

SEQ ID NO: 32 - protein, 7 aa, synthetic construct
TVDTKLL

SEQ ID NO: 33 - protein, 5 aa, synthetic construct
INLAF

SEQ ID NO: 34 - protein, 5 aa, synthetic construct
VKGSA

SEQ ID NO: 35 - protein, 5 aa, synthetic construct
RRFVN

SEQ ID NO: 36 - protein, 5 aa, synthetic construct
SSTTS

SEQ ID NO: 37 - protein, 5 aa, synthetic construct
SLTLT

SEQ ID NO: 38 - protein, 5 aa, synthetic construct
RLYLT

SEQ ID NO: 39 - protein, 5 aa, synthetic construct
VGSMF

SEQ ID NO: 40 - DNA, 202 bp, synthetic construct
  source 1..202: mol_type = other DNA
  variation 148..152: note = n is any DNA residue; note = note = k is either G or T
gccatgggtaaatctccggaagaactgaaacgtatcttcgaaaaatacgctgctaaagaaggtgacccggaccagctgtctaaagacgaactgaaactgctgatccaggctgaattcccgtctctgctgaaaggtatgggtggcggtnnknnknnknnknnkggtggtggctctaccctggacgacctgttccaggaactgg

SEQ ID NO: 41 - DNA, 205 bp, synthetic construct
  source 1..205: mol_type = other DNA
  variation 148..165: note = n is any DNA residue; note = k is either G or T
gccatgggtaaatctccggaagaactgaaacgtatcttcgaaaaatacgctgctaaagaaggtgacccggaccagctgtctaaagacgaactgaaactgctgatccaggctgaattcccgtctctgctgaaaggtatgggtggcggtnnknnknnknnknnknnkggtggtggctctaccctggacgacctgttccaggaactgg

SEQ ID NO: 42 - DNA, 208 bp, synthetic construct
  source 1..208: mol_type = other DNA
  variation 148..168: note = n is any DNA residue; note = k is either G or T
gccatgggtaaatctccggaagaactgaaacgtatcttcgaaaaatacgctgctaaagaaggtgacccggaccagctgtctaaagacgaactgaaactgctgatccaggctgaattcccgtctctgctgaaaggtatgggtggcggtnnknnknnknnknnknnknnkggtggtggctctaccctggacgacctgttccaggaactgg

SEQ ID NO: 43 - DNA, 196 bp, synthetic construct
  source 1..196: mol_type = other DNA
  variation 142..162: note = n is any DNA residue; note = k is either G or T
gccatgggtaaatctccggaagaactgaaacgtatcttcgaaaaatacgctgctaaagaaggtgacccggaccagctgtctaaagacgaactgaaactgctgatccaggctgaattcccgtctctgctgaaaggtatgggtnnknnknnknnknnknnknnkggttctaccctggacgacctgttccaggaactgg

SEQ ID NO: 44 - DNA, 208 bp, synthetic construct
  source 1..208: mol_type = other DNA
  variation 148..167: note = n may be any DNA residue; note = k is either residues g or t; note = d is any of residues g, a or t; note = m is either residue a or c; note = y is either residue t or c; note = v is any of residues a, c or g; note = h is any of residues a, c or t
gccatgggtaaatctccggaagaactgaaacgtatcttcgaaaaatacgctgctaaagaaggtgacccggaccagctgtctaaagacgaactgaaactgctgatccaggctgaattcccgtctctgctgaaaggtatgggtggcggtdmtnytdmtnytmvanytvhgggtggtggctctaccctggacgacctgttccaggaactgg

SEQ ID NO: 45 - protein, 7 aa, synthetic construct
QPVVRTN

SEQ ID NO: 46 - protein, 7 aa, synthetic construct
YTNAISD

SEQ ID NO: 47 - protein, 7 aa, synthetic construct
KMRPGVN

SEQ ID NO: 48 - protein, 7 aa, synthetic construct
HFQWHPT

SEQ ID NO: 49 - protein, 7 aa, synthetic construct
WHHTRLE

SEQ ID NO: 50 - protein, 7 aa, synthetic construct
AVHHMPF

SEQ ID NO: 51 - DNA, 18 bp, synthetic construct
  source 1..18: mol_type = other DNA
  misc_feature 1..18: note = S100Gforward primer
ggccatggccaaatctcc

SEQ ID NO: 52 - DNA, 19 bp, synthetic construct
  source 1..19: mol_type = other DNA
  misc_feature 1..19: note = S100Greverse primer
ccagttcctggaacaggtc

SEQ ID NO: 53 - protein, 7 aa, synthetic construct
VNIGLEY

SEQ ID NO: 54 - protein, 7 aa, synthetic construct
LFVMTRM

SEQ ID NO: 55 - protein, 7 aa, synthetic construct
HHYTVFM

SEQ ID NO: 56 - protein, 7 aa, synthetic construct
ILALFFV

SEQ ID NO: 57 - protein, 7 aa, synthetic construct
EDHREMD

SEQ ID NO: 58 - protein, 7 aa, synthetic construct
HPRSTAV

SEQ ID NO: 59 - protein, 7 aa, synthetic construct
IMGYPLN

SEQ ID NO: 60 - protein, 7 aa, synthetic construct
FGVHEWV

SEQ ID NO: 61 - protein, 7 aa, synthetic construct
PTDIWAW

SEQ ID NO: 62 - protein, 7 aa, synthetic construct
PIHESEH

SEQ ID NO: 63 - protein, 7 aa, synthetic construct
IRQDAQA

SEQ ID NO: 64 - protein, 7 aa, synthetic construct
RHRKPFE

SEQ ID NO: 65 - protein, 7 aa, synthetic construct
GLDTRHD

SEQ ID NO: 66 - protein, 7 aa, synthetic construct
TLGKMHH

SEQ ID NO: 67 - protein, 7 aa, synthetic construct
KNMQMWV

SEQ ID NO: 68 - protein, 7 aa, synthetic construct
DYQPQGI

SEQ ID NO: 69 - protein, 7 aa, synthetic construct
WPVGHAT

SEQ ID NO: 70 - protein, 7 aa, synthetic construct
MKQGPVY

SEQ ID NO: 71 - protein, 7 aa, synthetic construct
FKRSWIF

SEQ ID NO: 72 - protein, 7 aa, synthetic construct
HITHNET

SEQ ID NO: 73 - protein, 7 aa, synthetic construct
QGKSVPA

SEQ ID NO: 74 - protein, 7 aa, synthetic construct
YLTIRLM

SEQ ID NO: 75 - protein, 7 aa, synthetic construct
ASNTYFS

SEQ ID NO: 76 - protein, 7 aa, synthetic construct
LIWGFKT

SEQ ID NO: 77 - protein, 7 aa, synthetic construct
PDPLDFD

SEQ ID NO: 78 - protein, 7 aa, synthetic construct
DLVSFYY

SEQ ID NO: 79 - protein, 7 aa, synthetic construct
SWMALLV

SEQ ID NO: 80 - protein, 7 aa, synthetic construct
SSWRGTT

SEQ ID NO: 81 - protein, 7 aa, synthetic construct
HHQMTKS

SEQ ID NO: 82 - protein, 7 aa, synthetic construct
PWTVPVD

SEQ ID NO: 83 - protein, 7 aa, synthetic construct
EGVNEFF

SEQ ID NO: 84 - protein, 7 aa, synthetic construct
IRWTVMM

SEQ ID NO: 85 - protein, 7 aa, synthetic construct
GYRWWWV

SEQ ID NO: 86 - protein, 7 aa, synthetic construct
DRSNSPE

SEQ ID NO: 87 - protein, 7 aa, synthetic construct
RAHDASI

SEQ ID NO: 88 - protein, 7 aa, synthetic construct
VHTKAAA

SEQ ID NO: 89 - protein, 7 aa, synthetic construct
DQWIEHV

SEQ ID NO: 90 - protein, 7 aa, synthetic construct
NEMFVVW

SEQ ID NO: 91 - protein, 7 aa, synthetic construct
DRQWYPA

SEQ ID NO: 92 - protein, 7 aa, synthetic construct
VHKFGHI

SEQ ID NO: 93 - protein, 10 aa, synthetic construct
QEELEALIDH

SEQ ID NO: 94 - protein, 10 aa, synthetic construct
SAELNSLIVA

SEQ ID NO: 95 - protein, 10 aa, synthetic construct
FFELNQLIFS

SEQ ID NO: 96 - protein, 10 aa, synthetic construct
IQELKYLIYH

SEQ ID NO: 97 - protein, 10 aa, synthetic construct
VIELVILIDD

SEQ ID NO: 98 - protein, 10 aa, synthetic construct
APELLHLIDE

SEQ ID NO: 99 - protein, 10 aa, synthetic construct
HKELSQLIWF

SEQ ID NO: 100 - protein, 10 aa, synthetic construct
PKELNRLIFG

SEQ ID NO: 101 - protein, 10 aa, synthetic construct
YGELGYLIHG

SEQ ID NO: 102 - protein, 10 aa, synthetic construct
EHELTLLILF

SEQ ID NO: 103 - protein, 10 aa, synthetic construct
YMELFILIAI

SEQ ID NO: 104 - protein, 10 aa, synthetic construct
SIELRHLIYE

SEQ ID NO: 105 - protein, 10 aa, synthetic construct
GDELHKLILY

SEQ ID NO: 106 - protein, 10 aa, synthetic construct
FWELNILIVY

SEQ ID NO: 107 - protein, 10 aa, synthetic construct
DQELEGLIHP

SEQ ID NO: 108 - protein, 10 aa, synthetic construct
SLELFPLIDD

SEQ ID NO: 109 - protein, 10 aa, synthetic construct
DAELNKLINP

SEQ ID NO: 110 - protein, 10 aa, synthetic construct
AYELPYLIIP

SEQ ID NO: 111 - protein, 10 aa, synthetic construct
IWELNILIEL

SEQ ID NO: 112 - protein, 10 aa, synthetic construct
AIELYALIFK

SEQ ID NO: 113 - protein, 10 aa, synthetic construct
DYELPNLIPP

SEQ ID NO: 114 - protein, 10 aa, synthetic construct
EFELGPLIEA

SEQ ID NO: 115 - protein, 10 aa, synthetic construct
GNELMQLIMM

SEQ ID NO: 116 - protein, 10 aa, synthetic construct
YYELDDLIMP

SEQ ID NO: 117 - protein, 10 aa, synthetic construct
TAELRNLIWA

SEQ ID NO: 118 - protein, 10 aa, synthetic construct
THELPILILS

SEQ ID NO: 119 - protein, 10 aa, synthetic construct
MAELHALIGY

SEQ ID NO: 120 - protein, 10 aa, synthetic construct
MRELVELIHV

SEQ ID NO: 121 - protein, 10 aa, synthetic construct
NEELEHLINE

SEQ ID NO: 122 - protein, 10 aa, synthetic construct
GPELYALIIF

SEQ ID NO: 123 - protein, 10 aa, synthetic construct
HNELPDLIQL

SEQ ID NO: 124 - protein, 10 aa, synthetic construct
HSELELLIEY

SEQ ID NO: 125 - protein, 10 aa, synthetic construct
VMELANLIGT

SEQ ID NO: 126 - protein, 10 aa, synthetic construct
HFELSRLINY

SEQ ID NO: 127 - protein, 10 aa, synthetic construct
WTELTVLIVW

SEQ ID NO: 128 - protein, 10 aa, synthetic construct
PTELQILIHW

SEQ ID NO: 129 - protein, 10 aa, synthetic construct
TPELAILISY

SEQ ID NO: 130 - protein, 10 aa, synthetic construct
PPELQNLIHF

SEQ ID NO: 131 - protein, 10 aa, synthetic construct
FLELDVLIHA

SEQ ID NO: 132 - protein, 10 aa, synthetic construct
YYELTELIYV

SEQ ID NO: 133 - protein, 10 aa, synthetic construct
RQELGFLILF

SEQ ID NO: 134 - protein, 10 aa, synthetic construct
KNELYWLIVE

SEQ ID NO: 135 - protein, 10 aa, synthetic construct
DSELKYLIYW

SEQ ID NO: 136 - protein, 10 aa, synthetic construct
DDELFMLIRM

SEQ ID NO: 137 - protein, 10 aa, synthetic construct
VIELWILIDD

SEQ ID NO: 138 - protein, 13 aa, synthetic construct
GGGYLTIRLMGGG